(19) United States
(12) Patent Application Publication
(10) Pub. No.: US 2023/0270149 A1
(43) Pub. Date: Aug. 31, 2023

(54) COMPOSITION

(71) Applicant: Givaudan SA, VERNIER (CH)

(72) Inventors: Michael LAGUERRE, Le Pontet (FR); Lena Lore HECHT, Wallisellen (CH); Francois-Xavier Henri PIERRE, Saint Saturnin les Avignon (FR); Samuel STENGEL, Meilen (CH); Mathieu TENON, Malemort du Comtat (FR); Rosa NAVARRO, Macul (CL); Céline GERIN, Orgon (FR); Victor VIARD, Avignon (FR)

(21) Appl. No.: 18/031,583

(22) PCT Filed: Oct. 12, 2021
(86) PCT No.: PCT/EP2021/078232

§ 371 Date: Apr. 12, 2023

(30) Foreign Application Priority Data
Oct. 13, 2020 (GB) 2016236.8

(51) Int. Cl. A23L 29/10; A23D 7/005; A23D 7/01; A23D 7/04; A23C 11/10; A61K 47/46

(52) U.S. Cl. CPC A23L 29/10; A23D 7/0053; A23D 7/011; A23D 7/04; A23C 11/103; A61K 47/46

(57) ABSTRACT

The present invention is related to extracts rich in polar lipids obtained or obtainable from macroalgae, microalgae, photosynthetic bacteria and/or photosynthetic organ(s) and/or tissue(s) of a plant and combinations thereof as well as their uses as emulsifiers.

### FIELD OF THE INVENTION

The present invention relates to natural emulsifying systems and uses thereof.

### BACKGROUND OF THE INVENTION

Emulsions are widely used in food technology, for instance as a means to improve the nutritional profile of food products by enabling fat content reduction, and/or the incorporation of water soluble nutrients and flavorings. Emulsions are normally obtained with the aid of different molecular emulsifying agents like emulsifiers, proteins or amphiphilic polymers (also called stabilizers). These ingredients are indispensable to the manufacture of stable commercially acceptable emulsion based products. Efficient stabilizer and emulsifier systems already exist, but these are often based on chemically modified ingredients. Emulsifiers and stabilizers are generally considered as additives which under many countries' health regulations must be declared in the product label by their respective E-numbers and some are considered “synthetic” ingredients, i.e. obtained by chemical processing. There is a growing demand from consumers for products which are free from artificial additives or so-called “E numbers”.

Thus, there is a need for replacing synthetic or artificial emulsifiers with natural emulsifier systems that can provide the necessary tensioactive properties whilst not compromising on the product quality.

Natural ingredients with emulsifying properties are known, but they are usually not as efficient as synthetic emulsifiers and/or present other drawbacks.

For example Quillaia is known for its emulsifying properties. However, this plant contains saponins which are toxic for humans at certain concentrations.

Oat oils are also used as emulsifiers, however their lipid composition is dominated by palmitic and linoleic (omega-6) acids, while consumers seek more omega-3 lipids such as alpha-linolenic acid in their food. Furthermore, oat does not contain gluten, but many people with coeliac disease avoid eating them because they often can become contaminated during the manufacturing process and/or transportation with other cereals that contain gluten such as stray wheat, rye, or barley.

The use of soy lecithins as emulsifiers is also widespread in the food sector, but these functional molecules suffer from many drawbacks, of which the negative impact on sustainability due to the deforestation necessary for the cultivation of soybeans is probably the most important. The fact that most soybeans are GMO is another issue, as well as the possible allergenicity and potential for hormone disruption in humans of this material Accordingly, there is an ongoing need to provide efficient solutions to answer this important demand for natural emulsifier systems, all the while providing good nutritional profiles.

It is an object of the present invention to provide a natural emulsifier system which can replace synthetic emulsifiers in food and cosmetic applications.

### SUMMARY OF THE INVENTION

It has surprisingly been found by the inventors that naturally-occurring extracts rich in polar lipids sourced from a photosynthetic part of the plant or macroalgae (such as photosynthetic organs or tissues), microalgae and photosynthetic bacteria can be used to replace conventional emulsifiers to effectively stabilize emulsions.

In a first aspect, the invention provides an Extract or “Extract of the invention” comprising polar lipids (such as an Extract rich in polar lipids) obtained or obtainable from macroalgae, microalgae, photosynthetic bacteria and/or photosynthetic organ(s) and/or tissue(s) of a plant and combinations thereof.

In a particular embodiment, the Extract of the invention may be a Crude extract or a Purified extract comprising polar lipids (such as a Crude extract or a Purified extract rich in polar lipids).

In another aspect, the invention provides an Extract of the invention (such as a crude extract of the invention and/or a Purified extract of the invention) for use as emulsifier.

In another aspect, the invention provides the Use of an Extract of the invention (such as a crude extract of the invention and/or a Purified extract of the invention) to stabilize emulsions.

In another aspect, the invention provides and Emulsion (Emulsion of the invention) comprising at least one Extract of the invention (such as a crude extract of the invention and/or a Purified extract of the invention) as an emulsifying agent.

In another aspect, the invention provides a Process for preparing an emulsion comprising:
a) mixing ingredients of an aqueous phase;
b) mixing ingredients of a lipid phase;
c) dispersing one or more Extract of the invention (such as a crude extract of the invention and/or a Purified extract of the invention) in one or both of the aqueous phase or the lipid phase; and
d) homogenizing the two phases to form an emulsion.

Advantageously, the stabilized emulsions of the invention do not require the addition of any other emulsifier.

In a further aspect, the invention relates to a food or beverage product for humans or animals, a nutritional supplement, a nutraceutical formulation, a fragrance or flavouring, a pharmaceutical or veterinary formulation, an oenological or cosmetic formulation comprising an emulsion of the invention.

In a further aspect, the invention relates to a food or beverage product for humans or animals, a nutritional supplement, a nutraceutical formulation, a fragrance or flavouring, a pharmaceutical or veterinary formulation, an oenological or cosmetic formulation comprising at least one Extract of the invention (such as a crude extract of the invention and/or a Purified extract of the invention) as an emulsifying agent.

The details, examples and preferences provided in relation to any one or more of the stated aspects of the present invention will be further described herein and apply equally to all aspects of the present invention. Any combination of the embodiments, examples and preferences described herein below in all possible variations thereof is encompassed by the present invention unless otherwise indicated herein, or otherwise clearly contradicted by context.

### DETAILED DESCRIPTION OF THE INVENTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The following text sets forth a broad description of numerous different embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims.

The section headings used herein are for organizational purposes only and are not to be construed as limiting the subject matter described. All documents, or portions of documents, cited in this application, including, but not limited to, patents, patent applications, articles, books, etc. are hereby expressly incorporated by reference for the portions of the document discussed herein, as well as in their entirety.

The present invention concerns the use of Extracts (extracts of the invention) obtained or obtainable from macroalgae, microalgae, photosynthetic organ(s) and/or tissue(s) of a plant, and/or bacteria rich in polar lipids as the emulsifier system for the stabilization of a water-in-oil or oil-in-water emulsion. By “emulsifier” or “emulsifier system” is to be understood at least one ingredient with tensioactive properties.

The extracts of the invention (obtained or obtainable from macroalgae, microalgae, photosynthetic organ(s) and/or tissue(s) of a plant, and/or bacteria) may be Crude extracts (Crude extracts of the invention) comprising polar lipids or Purified extracts comprising polar lipids (or purified extracts of the invention). In certain embodiments, the crude extracts of the invention or the purified extracts of the invention are rich in polar lipids.

Crude extracts of the invention rich in polar lipids and Purified extracts of the invention rich in polar lipids from macroalgae, microalgae, photosynthetic organ(s) and/or tissue(s) of a plant, and/or photosynthetic bacteria have been found to stabilize emulsion remarkably against coalescence. Even more surprisingly, the extracts of the invention comprising polar lipids (such as Crude extracts of the invention and/or Purified extracts of the invention rich in polar lipids) from photosynthetic plants, macroalgae, microalgae and/or bacteria have been found to produce emulsions stables at very broad pH ranges.

Extracts of the Invention

According to the present invention, there is provided an Extract obtained or obtainable from a macroalgae, microalgae, photosynthetic organ(s) and/or tissue(s) of a plant, and/or photosynthetic bacteria or mixtures thereof comprising polar lipids. In certain embodiments, the Extract of the invention is rich in polar lipids. In the present invention the “Extracts of the invention” may be also defined as “Emulsifier Extracts of the invention” because of their emulsifier properties.

As will be appreciated by the person skilled in the art, as used herein the term “obtainable from” means that the Extract may be obtained from microalgae, macroalgae, photosynthetic organ(s) and/or tissue(s) of a plant, and/or photosynthetic bacteria or may be isolated from the microalgae, macroalgae, photosynthetic organ(s) and/or tissue(s) of a plant, and/or photosynthetic bacteria or may be obtained from an alternative source, for example by chemical synthesis or enzymatic production. Whereas the term “obtained” as used herein, means that the Extract is directly derived from the microalgae, the macroalgae, the photosynthetic organ(s) and/or tissue(s) of a plant, and/or photosynthetic bacteria sources.

The Extract of the invention may be a Crude extract (or Crude extract of the invention) or a Purified Extract (Purified extract of the invention).

In a preferred embodiment, the plant, macroalgae, microalgae and bacteria are photosynthetic organisms. In one embodiment, the Extract of the invention (such as a Crude or Purified extract) is obtained or obtainable from a photosynthetic part of the plant or macroalgae such as photosynthetic organ(s) or tissue(s)) including but not limited to the leaves or the stems. In a preferred embodiment, the plant, microalgae, macroalgae and/or photosynthetic bacteria are recognized as food grade or recognized as GRAS (Generally Recognized as Safe). The Extract of the invention (such as a Crude or Purified extract) may be obtainable or may be obtained from one single source or from different sources (such as one or more photosynthetic organs or tissues from one or more plants, one or more microalgae, one or more macroalgae, or one or more photosynthetic bacteria or mixtures thereof).

The Extracts of the invention may be extracted using any methods known in the art. In certain embodiments, the material (such as the photosynthetic part of a plant, macroalgae, microalgae and/or photosynthetic bacteria) may be processed before extraction, for example it can be washed, dried, cut, milled or grounded, etc. Additionally enzymatic processing may be used to disrupt the cellulose walls as. For example the material may be contacted with an enzyme preparation having cellulase, β-glucanase or β-glucosidase activity; allowing the enzyme preparation to degrade the cell walls of the material.

The inventors have also surprisingly found that the starting material used may be fresh (wet material) or may be a dry material without modifying the emulsifying properties of the Emulsifier Extracts of the invention (see examples 33 to 36).

Thus, in certain embodiments, the starting material (such as the photosynthetic part of a plant, macroalgae, microalgae and/or photosynthetic bacteria) may be a fresh (or wet material). In certain embodiments, the starting material (such as the photosynthetic part of a plant, macroalgae, microalgae and/or photosynthetic bacteria) may be a dry or partially dry materia. Dry material is understood as a material where at least 90% of the water was evaporated. Partially dried materiel is understood herein as a material where at least 30% of the water was evaporated, such as at least 50% of the water was evaporated. In certain embodiments for example when the material is very rich in water, the juice from the material (such as spinach) may be filtrated and the rest (filtration cake) is used for the extraction (see for example 32)

The starting material (such as the photosynthetic part of a plant, macroalgae, microalgae and/or photosynthetic bacteria) may be a “raw material” or a “spent material”.

The term “raw material” as used herein, refers to one or more of the above mentioned material that was not used before for other extraction process (for example whole spirulina with all the components present naturally in the spirulina cells).

The term “spent material” as used herein, refers to one or more of the above mentioned materials that was used already for the extraction of another product and that is recovered and processed again for extracting the Emulsifier Extract of the invention (such as a crude or purified extract rich in polar lipids). Said spent material is not toxic and comprises most of the components present naturally in the original material, at least it comprises most of the polar lipids originally present in the starting material (such as spirulina, macroalgae, microalgae etc). For example, the Emulsifier Extract of the invention (such as a crude or purified extract rich in polar lipids) may be obtained from spirulina cakes that were used for extraction of other materials (such as colours, etc), spinach cakes that were used for extraction of other materials etc. Such cakes may be, for example, the resulting material after the raw original material has being treated with a solvent to extract other product(s) different to polar lipids and then separated from said solvent and eventually dried. In one embodiment, the material is spent material from photosynthetic bacteria such as spirulina, from microalgae such as Chlorella (such as Chlorella sorokiniana, Chlorella vulgaris, Chlorella zofingensis), Chysophyceae, Xantophyceae, Baccilariophyceae, Dinophyceae, Rodophyceae, Phaeophyceae, Chlorophyceae, Prasinophyceae, Cryptophyceae, Crypthecodinium, Cylindrothec, Botrycoccus, Dunaliella (such as Dunaliella salina), Euglena gracilis, Isochrysis, Tetraselmis, Nannochloropsis, Neochloris, Nitzschia Scenedesmus, Chlorobotrys, Eustigmatos, Phaeodactylum, Porphyridium, Pseudostaurastrum, Schizochytrium, Tetraselmis, Vischeria, Monodopsis, Ellipsoidion,

Pseudocharaciopsis, from macroalgae such as Ulva spp or from a photosynthetic part of a plant, such as spinach leaves and stems.

An example of extraction method for obtaining a Crude extract may comprise:
a) Mixing the material (such as the photosynthetic part of a plant, macroalgae, microalgae and/or photosynthetic bacteria) with a solvent
b) Separating the solvent from residual material by any suitable separation technique known in the art
c) separating the solvent partially or totally from the Extract
d) Optionally drying the crude Extract.

Particular solvents that may be used in the extraction process include water, alcohols (such as methanol, ethanol, isopropanol), acetone, ethyl acetate, hexane, dichloromethane, 2-methyltetrahydrofuran, chloroform (such as >98%, stabilized with 0.6% ethanol) and any mixtures thereof, such as alcohol:water mixtures (such as mixtures of methanol and water, or ethanol and water, or isopropanol and water) or acetone:water mixtures. For example, the extraction solvents can be a water:alcohol mixture (from about 50% to about 99% alcohol in water. For example, from about 60% to about 90% alcohol in water, or from about 70% to about 80% alcohol in water), or a pure alcohol. Particular alcohols that may be mentioned include ethanol (EtOH), methanol (MeOH), and isopropanol (iPrOH).

In particular embodiments, the extraction solvent may be a methanol:water mix, such as from about 50% to about 99% methanol in water, or from about 60% to about 90% methanol in water. For example, from about 70% to about 80% methanol in water.

The term “acetone crude extract” as used herein, refers to the Crude Extract obtained from any of the material mentioned before (such as the photosynthetic part of a plant, macroalgae, microalgae and/or photosynthetic bacteria) when the extraction from the material (such as spinach leaves, yerba mate, Agarophyton chilensis, Ulva sp., spirulina, Nannochloropsis sp., Chlorella sp., Dunaliella salina, etc.), has been performed using acetone as the only solvent or with a mixture of acetone and water (such as from about 60% acetone to 90% acetone in water, such as 70:30, 80:20 or 90:10 acetone:water). The term ‘hydro-acetonic crude extract’ as used herein, refers to the Crude Extract obtained from any of the material mentioned before (such as spinach leaves, yerba mate, Agarophyton chilensis, Ulva sp, spirulina, Nannochloropsis sp., Chlorella sp., Dunaliella, etc.) when the extraction from the biological material has been performed using a mixture of water and acetone. For example, from about 1% to about 99% acetone in water, such a crude extract would be termed a hydro-acetonic crude extract.

The term ‘alcohol crude extract’ or ‘alcoholic crude extract’ as used herein, refers to the Crude Extract obtained from any of the material mentioned before (such as the photosynthetic part of a plant, macroalgae, microalgae and/or photosynthetic bacteria) when the extraction has been performed using alcohol as the only solvent. For example, 100% methanol and/or 100% ethanol (absolute ethanol). The term ‘hydro-alcoholic crude extract’ as used herein, refers to the Crude Extract obtained from any of the material mentioned before (such as spinach leaves, yerba mate, Agarophyton chilensis, Ulva sp, spirulina, Nannochloropsis sp., Chlorella sp., Dunaliella, etc.) when the extraction from the biological material has been performed using a mixture of water and alcohol. For example, from about 1% to about 99% alcohol (e.g. ethanol, methanol, isopropanol) in water, such a crude extract would be termed a hydroethanolic crude extract.

The term “hexane crude extract” or “hexanic crude extract” as used herein, refers to the Crude Extract obtained from any of the biological material mentioned before when the extraction has been performed using hexane as the only solvent.

The term “ethyl acetate crude extract” as used herein, refers to the Crude Extract obtained from any of the biological material mentioned before when the extraction has been performed using ethyl acetate as the only solvent.

The term “methyltetrahydrofuran crude extract” as used herein, refers to the Crude Extract obtained from any of the biological material mentioned before when the extraction has been performed using methyltetrahydrofuran as the only solvent.

Extractions using mixtures of one or more of the above mentioned solvents can be used (such as for example chloroform/methanol mixtures, etc.)

In one embodiment, the temperature of extraction is in a range of from about 20° C. to about 100° C. In a particular embodiment, the temperature for extraction is in a range of from about 50° C. to about 70° C. Typically, the ratio of biological material to solvent mixture used in the extraction process varies from about 1:1 to about 1:74 on a gram to milliliter basis, such as from about 1:10 to about 1:20. The incubation period (i.e. the period during which the biological material is in contact with the solvent) is typically from about 1 hour to about 24 hours.

Mechanical energy can be applied during the extraction process. Applying mechanical energy helps to homogenize the mixture, changes the physical structure of the starting biological material and increases the extraction yields of polar lipids. The amount of mechanical energy applied in the method depends on at which step applied, the type of material, the amount of the starting material used in the mixture, the pH of the mixture, and the temperature of the mixture. The amount of mechanical energy also can influence the amount of time needed to complete the extraction.

For example, the material (such as photosynthetic parts of a plant and/or a microalgae) and the extraction solution (such as acetone or ethanol) may be mixed using techniques known in the art, for example using stirring, shearing, maceration, percolation or infusion, such as magnetic or mechanical stirring. Stirring may be conducted at any suitable revolution per minute (rpm), for example, the stirring may be done from about 1 rpm to about 10 rpm or from about 50 rpm to about 500 rpm. For mechanical stirring this may typically be done from about 1 rpm to 500 rpm, such as from about 10 rpm to about 200 rpm. Devices for applying mechanical energy can be a pump, a refiner, a rotor-stator, a homogenizer, an extruder, a lobe pump, and/or a centrifugal pump. The mixture can be circulated in a closed-loop system that includes a pressure vessel (able to contain a heated solvent mixture), a reflux vessel, a heat exchanger, such as a shell and tube heat exchanger, and a pump for recirculating the heated mixture back to the vessel, allowing multiple passes through the pump in the system. In certain embodiments, reflux may be used.

After the photosynthetic material (such as spinach leaves, spirulina, etc.) and the solvent have been incubated, the solvent is separated from residual material by any suitable separation technique known in the art (like for example filtration, such as AF06 or AF31H filter plates).

Further filtration steps can be used to ensure the resulting Crude Extract is devoid of any potential solid particles coming from the biological material.

The solvent may be partially or totally separated from the extract by any method known in the art such as evaporation at atmospheric pressure or at reduced pressure, distillation, and any device allowing solvent evaporation, or distillation, or a liquid-liquid way of replacing the solvent. Pasteurisation or sterilisation methods can be applied at any step of the extraction.

In one embodiment, the Crude extract comprising polar lipids of the invention is rich in polar lipids. “Enriched in polar lipids” or “rich in polar lipids” in the present invention and in the specific context of crude extracts, means that the crude extract of the invention comprises at least 5 wt %, at least 10 wt %, at least 11 wt %, at least 12 wt %, at least 13 wt %, at least 14 wt %, at least 15 wt %, at least 16 wt %, at least 17 wt %, at least 18 wt %, at least 19 wt %, at least 20 wt %, at least 25 wt %, at least 30 wt %, at least 35 wt %, at least 40 wt %, at least 45 wt %, at least 50 wt % of polar lipids in relation to the total extract.

The crude extracts of the invention can further contain other natural substances such as sugars, proteins, peptides, chlorophylls or waxes, etc.

In particular embodiments, the Crude extracts of the invention may be:
substantially free of other biological material (e.g. free of plant cellulose debris);
substantially free of plant, algae or bacteria cells; and/or
substantially free of plant, algae or bacteria cellular matter,
substantially free of toxic components like pesticides, quintozene, aflatoxins, ochratoxin A, cadmium, arsenic, lead or mercury, and/or
substantially free of residual solvents.

As used herein, references to an extract being “substantially free” of another biological material debris or undesirable natural substances may refer to the extract consisting of less than 1% by weight (e.g. less than 0.1%, such as less than 0.01% or less than 0.001%, by weight) of that other material.

The Crude extracts of the invention can be further purified to obtain extracts more enriched in polar lipids and/or depleted of non-desirable substances so as to obtain a “Purified Extract” (or Purified Extract of the invention). In certain embodiments, the Purified Extracts of the invention is rich in polar lipids. Any method known in the art that allows further concentration of the polar lipids fraction of the extract and/or that allows partial or total depletion of non-emulsifying agents or other substances that may affect the emulsifying properties of the Emulsifier Extracts of the invention and/or the organoleptic properties of said extracts, may be used in the present invention.

For example during the production of the crude extract, before step b) (separation of the solvent from the residual the solvent) a second liquid/liquid extraction may be performed followed by an equilibration and recovering of the phase containing the polar lipids and other emulsifying agents. Further, the Crude Extracts of the invention can be also processed as to obtain Purified extracts that are depleted in substances that may interfere in the emulsifying properties of the Extract such as sugars, proteins, peptides, chlorophylls, waxes, etc.

The purified Extract of the invention may be obtained using further purification methods known in the art including but not limited to one or more of: use of charcoal (such as powdery activated carbon or carbon filter plate), absorption on solid phase, chromatographic methods, resins, membrane separation, membrane purification, solid/liquid extraction using polar or apolar solvents, liquid/liquid separation using polar or apolar solvents, further centrifugation steps, distillation, fractional distillation, molecular distillation, striping with water, etc.

In certain embodiments, the purification is done using an activated carbon filter that may be used after the filtration step and/or during the filtration step.

In certain embodiments, activated carbon is used to purify the extracts. For example the activated carbon may be added to the filtrate. The mixture may then be heated to about 50° C. and a new filtration may be performed with a normal filter (such as a AF31H filter plate).

In a preferred embodiment, the purified extracts of the invention contain less than 10% of sugars, proteins, peptides, chlorophylls and/or waxes, or preferably less than 5% of them, or more preferably less than 1% of them, such as less than 0.01% of them.

In one embodiment, the purified extract rich in polar lipids (purified extract rich in polar lipids of the invention) comprises at least 5 wt %, such as at least 10 wt %, at least 15 wt %, at least 20 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, at least 99 wt % of polar lipids in relation to the total extract components.

In a preferred embodiment, the Purified Extract of the invention contain less than 10% of sugars, proteins, peptides, chlorophylls and/or waxes, or preferably less than 5% of them, or more preferably less than 1% of them, such as less than 0.01% of them, and comprises at least 15% wt %, at least 20 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, at least 99 wt % of polar lipids in relation to the total weight of the extract.

In particular embodiments, the Purified extracts of the invention may be:
substantially free of other biological material (e.g. free of plant cellulose debris);
substantially free of plant, algae or bacteria cells; and/or
substantially free of plant, algae or bacteria cellular matter,
substantially free of toxic components like pesticides, quintozene, aflatoxins, ochratoxin A, cadmium, arsenic, lead or mercury,
substantially free of residual solvents,
substantially free of chlorophylls, and/or
substantially free of sugars, proteins, peptides, and/or waxes,
substantially free of off-notes,
substantially free of green, brown or grey colour.

In particular embodiments, the Purified extracts of the invention have improved organoleptic properties if compared with crude extracts. In the present invention, “improved organoleptic properties” means that the purified extract of the invention has not a negative odour, has not a dark colour (such as green or brown colour) and/or has not an off-taste that may impact the appearance and performance of the Emulsifier extract of the invention in the final application.

In certain embodiments, the purified extract is decolorized. As “decolorized” in the present invention is understood as an Extract where the dark colour (such as green, black or brown colour) is removed to obtain a purified extract with more light colours (transparent, white or light yellow). In certain embodiments, the purified extract has a L₁*, a₁*, and b₁* that corresponds to transparent, white or light yellow or is close to transparent, white or light yellow.

Thus, the invention also relates to purified extracts rich in polar lipids obtained or obtainable from photosynthetic parts of plants (such as spinach and/or alfalfa leaves or stems), macroalgae, photosynthetic bacteria or mixtures thereof as defined previously.

The Extracts of the invention may be obtained or obtainable from the whole plants or from photosynthetic organ(s) and/or tissue(s) of the plants.

Examples of plants that may be used in the present invention include but is not limited to plants from the Rutaceae family (including but not limited to Citrus such as orange, lime or lemon), the Malvaceae family (including but not limited to cocoa and marshmallow), the Rubiaceae family (including but not limited to coffee), the Amaranthaceae family (including but not limited to beetroot and spinach), the Poaceae family (including but not limited to bamboo and oat), the Zingiberaceae family (including but not limited to curcuma), the Ginkgoaceae (including but not limited to ginkgo), the Araliaceae family (including but not limited to ginseng), the Theaceae (including but not limited to matcha tea), the Asteraceae family (including but not limited to milk thistle), the Oleaceae family (including but not limited to olive tree), the Moringaceae family (including but not limited to moringa), the Bromeliaceae family (including but not limited to pineapple), the Brassicaceae family (including but not limited to broccoli rabe, broccoli, red radish), the Rosaceae family (including but not limited to rosehip), the Sapindaceae family (including but not limited to guarana), and the Lamiacea family (including but not limited to rosemary, sage, thyme, mint, basil, Perilla frutescens and oregano), the Apiaceae family (including but not limited to ajwain, angelica, anise, asafoetida, caraway, carrot, celery, chervil, coriander, cumin, dill, fennel, lovage, cow parsley, parsley, parsnip and sea holly, as well as silphium), Asteraceae family (including but not limited to lettuce), Fabaceae family (including but not limited to alfalfa, and the genus Phaseolus, chickpea Cicer arietinum, fenugreek (Trigonella foenum-graecum), lentil (Lens culinaris), lupine (genus Lupinus), pea (Pisum sativum), etc.), yerba mate (Ilex paraguariensis), genus Cichorium (including but not limited to endive), nettle (Urtica dioica), Amaryllidaceae family (including but not limited to garlic, scallion, leek, chive, and chinese onion, onions, green onions) and mixtures thereof. In certain embodiments, the Emulsifier Extract of the invention (such as a crude or purified extract comprising polar lipids) is obtained or obtainable from the whole plant. In certain embodiments, the Emulsifier Extract of the invention (such as a crude or purified extract comprising polar lipids) is obtained or obtainable from the photosynthetic organs or tissues like the leaves, stems, etc. of said plant (also defined herein as “greens”). In certain preferred embodiments, the Extracts of the invention obtained or obtainable from greens of plants (greens plant Extracts of the invention) include but is not limited to greens of: broccoli rabe, broccoli, red radish, guarana, rosemary, sage, thyme, mint, basil, Perilla frutescens, ajwain, angelica, anise, asafoetida, caraway, carrot, celery, chervil, coriander, cumin, dill, fennel, lovage, cow parsley, parsley, parsnip, sea holly, silphium oregano, lettuce, alfalfa fenugreek (Trigonella foenum-graecum), lentil (Lens culinaris), lupine (genus Lupinus), pea (Pisum sativum), garlic, scallion, leek, chive, chinese onion, onions, green onions, beetroot, spinach, parsley, yerba mate, tea, endive, watercress, nettle, carrots, sweet potato (Ipomoea batatas) leaves, pak choi (Brassica rapa subsp. chinensis), water spinach (Ipomoea aquatica) and mixtures thereof, and optionally the solvent is selected from alcohol or alcohol water mixtures, such as ethanol 100% or ethanol:water (70:30, 80:20 or 90:10), and optionally the lipid contain is from at least 5%, such as least 10% of polar lipids. In certain embodiments, the Extract (crude or purified extract) is an extract obtained or obtainable from the greens of spring onion and is extracted using ethanol 100%.

In certain embodiments, the Extract (crude or purified extract) is an extract obtained or obtainable from alfalfa using ethanol-water as solvent (such as 80:20 or 90:10 ethanol:water). In certain embodiments, the alfalfa extract also comprises saponins.

The inventors have surprisingly found that greens of broccoli rabe, green pea pods, rosemary, celery, carrot, parsley, radish leaves and/or black tea leaves have very high emulsification properties even at low pH (see examples 30, 31 and 32).

In certain embodiments, the Extract (crude or purified extract) is an extract obtained or obtainable from the greens of broccoli rabe, green pea pods, rosemary, celery, carrot, parsley, radish leaves and/or black tea leaves and is extracted using ethanol 100% or ethanol-water (such as 90:10 or 80:20) as solvent.

The inventors have surprisingly found that spinach crude extracts or spinach purified extracts have very high emulsification properties even at low pH and low concentrations as well as high contain in polar lipids (see examples 1 to 6, 36, 41).

In one embodiment, the extract is a spinach extract obtained using ethanol or hydro-ethanol as solvent (such as 50:50 to 90:10 ethanol:water). In certain embodiments, the temperature of extraction is ambient temperature. In other embodiments, the temperature of extraction is from about 40° C. to about 100° C.

In certain embodiments, the spinach extract is a hydro-ethanol extract (90:10 EtOH:water) and the polar lipid contain is of at least 20%, such as at least 30%. In certain embodiments, the extract is a hydroethanol extract (90:10 EtOH:water) and wherein extraction is done at a temperature of least 40° C., such as 60° C., and, optionally, the polar lipid contain is of at least 40%, such as at least 45%. In certain embodiments, the spinach extract is an ethanol extract (100% EtOH) and, optionally, the polar lipid contain is of at least 30%, such as at least 34%.

In certain embodiments, the spinach extract is a hydro-acetone extract (90:10 acetone:water) and, optionally, the polar lipid contain is of at least 20%, such as at least 40% such as at least 65%, such as 69%.

In certain embodiments, the spinach extract is obtained using acetate as solvent and, optionally, the polar lipid contain is of at least 50%, such as at least 70%.

In certain embodiments, the spinach extract is obtained using isopropanol as solvent and, optionally, the polar lipid contain is of at least 20%, such as at least 30%. In certain embodiments, the spinach extract is obtained using MeTHF as solvent and, optionally, the polar lipid contain is of at least 20%, such as at least 30%.

In certain embodiments, the spinach may be fresh or it may be dried previous to the extraction process. The Spinach extracts mentioned herein may be crude or may be purified extracts as defined previously. The Spinach extracts mentioned herein may be obtained from fresh material or from dry material. The inventors have found that spinach extracts as described in the present invention comprise polar lipids. However, also some extracts with lower % of polar lipids have very good emulsifications properties; therefore in certain embodiments, the Spinach Extracts of the invention comprise polar lipids and optionally other emulsifying agents such as saponins, rhamnolipids, DAG.

Algae are photosynthetic eukaryotic organisms that include species from multiple distinct clades and includes organisms range from unicellular microalgae, such as Chlorella and the diatoms, to multicellular macroalgal forms such as seaweeds or freshwater algae.

Examples of macroalgae that may be used in the present invention are Ascophyllum nodosum; Fucus serratus, F. vesiculosus, Himanthalia elongate, Undaria pinnatifida, Laminaria digitata, L. saccharina, L. japonica, Alaria esculenta, Palmaria palmata (dulse), Porphyra umbilicalis; P. tenera, P. yezoensis, P. dioica, P. purpurea, P. laciniata, P. leucostica, Chondrus crispus; Gracilaria verrucosa, Lithothamnium calcareum, Enteromorpha spp. and Ulva spp.

Microalgae are eukaryotic, unicellular organisms ranging in size from few to hundreds of micrometers. The term ‘microalgae’ include highly diverse groups such as green algae, diatoms, dinoflagellates, and coccolithophores, and consist of an unknown number of species estimated to be tens or even hundreds of thousands.

The inventors have surprisingly found that microalgae extracts as described in the present invention can stabilize emulsions at low pH (pH 3.5) and high pH (pH 7) even at low concentrations. The inventors of the present invention have surprisingly found that the microalgae extracts as described in the present invention have a high contain in polar lipids (see Tables 6, 7, 8, 9 and 10).

Thus, in certain embodiments, the Emulsifier Extract of the invention is obtained or obtainable from microalgae (“microalgae extract of the invention”). In certain embodiments, the microalgae are green microalgae. In certain embodiments, the Emulsifier microalgae Extract of the invention (such as a crude or purified extract comprising polar lipids or such as a crude or purified extract rich in polar lipids) is obtained or obtainable from the microalgae including but not limited to Chlorella (such as Chlorella vulgaris, Chlorella sorokiniana, Chlorella zofingensis), Chysophyceae, Xantophyceae, Baccilariophyceae, Dinophyceae, Rodophyceae, Phaeophyceae, Chlorophyceae, Prasinophyceae, Cryptophyceae, Crypthecodinium, Cylindrothec, Botrycoccus, Dunaliella (such as Dunaliella salina), Euglena gracilis, Isochrysis, Nannochloropsis, Neochloris, Nitzschia Scenedesmus, Chlorobotrys, Eustigmatos, Phaeodactylum, Porphyridium, Pseudostaurastrum, Schizochytrium, Tetraselmis, Vischeria, Monodopsis, Ellipsoidion, Pseudocharaciopsis and combinations thereof.

In certain embodiments the microalgae extract of the invention is extracted using a solvent that is selected from ethyl acetate, isopropanol, acetone, chloroform:methanol (such as 2:1), methanol, MeTHF, ethanol and/or hydro-ethanol.

In certain embodiments, the microalgae extract of the invention (such as a Tetraselmis Extract, Nannochloropsis, Chlorella, Dunaliella, Isochrysis extract) may be a crude extract or a purified extract rich in polar lipids, and optionally comprises other emulsifying agents (such as saponins, etc.). In a preferred embodiment, microalgae extract (crude or purified extract) has at least 10% of polar lipids, at least 20% of polar lipids, at least 25% wt of polar lipids, at least 50% of polar lipids, such as at least 70%, such as at least 90% of polar lipids based on the total weight of the dry extract.

In certain embodiments, the Tetraselmis extract (crude or purified) is obtained using isopropanol, ethylacetate, and/or acetone as solvent and optionally, has a the polar lipid contain of at least 50%, such as at least 60%, such as at least 70%, such as at least 80%, such as at least 90%, such as 98%.

In certain embodiments, the Tetraselmis extract (crude or purified) is obtained using ethanol or ethanol and water (such as 100% ethanol or 90:10 EtOH:water) as solvent and, optionally, has a polar lipid contain of at least 50%, such as at least 60%, such as at least 70%, such as at least 80%, such as at least 90%, such as 69%. In certain embodiments, the Nannochloropsis extract (crude or purified) is obtained using ethanol or ethanol and water (such as 100% ethanol, 90:10, 80:20 or 70:30 EtOH:water) as solvent and, optionally, has a polar lipid contain of at least 20%, of at least 50%, such as at least 60%, such as at least 70%, at least 80% or at least 90%.

In certain embodiments, the Nannochloropsis extract (crude or purified) is obtained using isopropanol, acetone and/or ethylacetate as solvent and, optionally, has a polar lipid contain of at least 30%, such as at least 60%, such as at least 70%, such as at least 80%.

In certain embodiments, the Chlorella extract (crude or purified) is from Chlorella vulgaris, Chlorella zofingensis and/or Chlorella sorokiniana, and is obtained using Ethanol or ethanol and water (such as 100% ethanol, 90:10, 80:20 or 70:30 EtOH:water) as solvent and, optionally, has a polar lipid contain of at least 40%, such as at least 50%, such as at least 70%, at least 80% or at least 90%.

In certain embodiments, the Chlorella extract (crude or purified) is from Chlorella vulgaris, Chlorella zofingensis and/or Chlorella sorokiniana) and is obtained using isopropanol, acetone and/or ethylacetate as solvent and, optionally, the polar lipid contain is of at least 30%, such as at least 60%, such as at least 70%, such as at least 80%. In certain embodiments, the Isochrysis extract (crude or purified) is obtained using ethanol or ethanol and water (such as 100% ethanol, 90:10, 80:20 or 70:30 EtOH:water) as solvent and, optionally, the polar lipid contain is of at least 20%, such as at least 50%, such as at least 70%, at least 80% or at least 90%.

In certain embodiments, the Isochrysis extract (crude or purified) is obtained using isopropanol, acetate and/or acetone as solvent and, optionally, the polar lipid contain is of at least 20%, such as at least 50%, such as at least 70%, at least 80% or at least 90%.

In certain embodiments of the microalgae Extracts described herein, additionally comprises other emulsifying agents (such as saponins, etc.).

In certain embodiments, the microalgae (such as Chlorella) may be grown under photo-autotrophy (photosynthesis), mixotrophy and heterotrophy.

In certain embodiment, the Emulsifier Extract of the invention (such as a crude or purified extract comprising polar lipids) is obtained or obtainable from photosynthetic bacteria including but not limited to cyanobacteria such as spirulina (Spirulina platensis, Spirulina maxima, Arthrospira platensis, Arthrospira maxima), Limnospira platensis, Klamath algae (Aphanizomenon flosaquae) and combinations thereof. In one embodiment, the Emulsifier extract of the invention is obtained or is obtainable from photosynthetic bacteria. In certain embodiments, the photosynthetic bacteria are cyanobacteria like spirulina (such as Spirulina platensis, Spirulina maxima also named Arthrospira platensis or Arthrospira maxima), Limnospira (Limnospira platensis), Synechocystis, Nostoc, Cyanothece and/or Aphanizomenon (such as Aphanizomenon flosaquae) and/or Klamath algae (Aphanizomenon flosaquae) and the solvent is selected from ethyl acetate, isopropanol, acetone, chloroform:methanol (such as 2:1), methanol, MeTHF, ethanol and/or hydroethanol. In certain embodiments, the photosynthetic bacteria Extracts of the invention (such as a spirulina extract) is rich in polar lipids.

In a preferred embodiment, the photosynthetic bacteria Extract of the invention (crude or purified extracts) have at least 5%, at least 10% of polar lipids, at least 20% of polar lipids, at least 30%, at least 40%, at least 50%, at least 60% of polar lipids based on the total weight of the dry extract. In a preferred embodiment, the photosynthetic bacteria is Spirulina spp. (also known as) and the solvent is selected from methyl tetrahydrofuran, hexane, isopropanol, ethanol or hydro-ethanol (such as 50:50, 60:40, 70:30, 80:20 or 90:10 ethanol water) or mixtures thereof.

The inventors of the present invention have surprisingly found that spirulina extracts (such as crude or purified extracts) obtained with polar solvents have very high emulsifications properties at broad ranges of pH (such as from 3 to 7 pH) (see examples 9, 10, 11, 12 and 13)

In certain embodiments, the Spirulina extract (crude or purified) is obtained using Ethanol or ethanol and water (such as 100% ethanol, 90:10, 80:20, 70:30, EtOH:water) as solvent and, optionally, the polar lipid contain is of at least 15%, at least 30%, at least 40%, at least 50%, at least 70%, at least 80% or at least 90%.

In certain embodiments, the spirulina extract (crude or purified) is obtained using hexane, acetone and/or MeTHF as solvent and, optionally, the polar lipid contain is of at least 15%, at least 30%, at least 40%, at least 50%, at least 70%, at least 80% or at least 90%.

In certain embodiments, the Spirulina extract (crude or purified) is obtained using isopropanol as solvent and, optionally, the polar lipid contain is of at least 15%, at least 30%, at least 40%, at least 50%, at least 70%, at least 80% or at least 90%.

In some embodiments, the spirulina extract is obtained using ethanol and water as solvent in a ratio from 30:70 to 70:30 EtOH:water.

In one embodiment, the biological material is selected from macroalgae (such as Ascophyllum nodosum, Fucus serratus, F vesiculosus, Himanthalia elongata, Undaria pinnatifida, Laminaria digitata, L. saccharine, L. japonica, Alaria esculenta, Palmaria palmata (dulse), Porphyra umbilicalis, P. tenera, P. yezoensis, P. dioica, P. purpurea, P. laciniata, P. leucostica, Chondrus crispus, Gracilaria verrucosa, Lithothamnium calcareum, Enteromorpha spp., Ulva spp., or mixtures thereof) and the solvent is selected from ethyl acetate, isopropanol, acetone and/or hydro-acetone, chloroform:methanol (such as 2:1), methanol, MeTHF, ethanol and/or hydro-ethanol.

In one embodiment, the macroalgae Emulsifier extract of the invention (crude or purified extract) (such as Ulva spp and/or Agarophyton chilensis extract) comprises polar lipids and optionally other emulsifying agents. In certain embodiments, the macroalgae Emulsifier extract of the invention (crude or purified extract) is rich in polar lipids.

In a preferred embodiment, the macroalgae Emulsifier extract of the invention has at least 5% of polar lipids, such as at least 10%, at least 20% of polar lipids or at least 25% of polar lipids, such as at least 45% of polar lipids based on the total weight of the dry extract. In a preferred embodiment, the macroalgae is Ulva spp and/or Agarophyton chilensis and the extracts are selected from hydro-ethanolic extracts (such as 90:10 ethanol:water), acetone or hydroacetonic crude extracts, or mixtures thereof. In a preferred embodiment, the extract is obtained from Ulva sp using 90:10 EtOH:water, and optionally the polar lipid concentration is of more than 30%, such as more than 40%.

Polar lipids include galactosyl acylglycerols (such as digalactosyldiacylglycerol (DGDG), monogalactosyldiacylglycerol (MGDG), digalactosylmonoacylglycerol (DGMG), or monagalactosylmonoacylglycerol (MGMG)), phospholipids (such as phosphatidylcholine (PC), phosphatidylethanolamine (PE) or phosphatidylglycerol (PG)), lysophospholipids (the monoacyl forms of PC, PE, or PG), sulphur lipids (such as sulfoquinovosyldiacylglycerol (SQDG)), betain lipids, furan-based lipids, or oxidation products of them like all above mentioned polar lipids containing at least one oxidized groups such as epoxide, peroxide, ketone or hydroxyl groups.

In particular embodiments, the Emulsifier Extracts of the invention (crude or purified extracts), comprises one or more of: galactosyl acylglycerols (such as digalactosyldiacylglycerol (DGDG), monogalactosyldiacylglycerol (MGDG), digalactosylmonoacylglycerol (DGMG), or monagalactosylmonoacylglycerol (MGMG)), phospholipids (such as phosphatidylcholine (PC), phosphatidylethanolamine (PE) or phosphatidylglycerol (PG)), lysophospholipids (the monoacyl forms of PC, PE, or PG), sulphur lipids (such as sulfoquinovosyldiacylglycerol (SQDG)), betain lipids, furan-based lipids, or oxidation products of them like all above mentioned polar lipids containing at least one oxidized groups such as epoxide, peroxide, ketone or hydroxyl groups. The Emulsifier Extracts of the invention may comprises other oils or lipids that are not polar lipids. The polar lipids and the non-polar lipids are named here as the “oil fraction”.

In particular embodiments of the above mentioned Emulsifier Extracts of the invention (crude or purified extracts), the polar lipid phase comprises at least 5 wt % of galactosyl acylglycerols, in another embodiment at least 8 wt %, for example at least 10 wt %, at least 11 wt %, at least 12 wt %, at least 20%, or at least 30% of galactosyl acylglycerols, based on the total weight of the oil fraction.

In particular embodiments, of the above mentioned Emulsifier Extracts of the invention (crude or purified extracts), the polar lipid phase comprises at least 5 wt % of galactosyl acylglycerols, in another embodiment at least 8 wt %, for example at least 10 wt %, at least 11 wt %, at least 12 wt %, at least 20%, or at least 30% of galactosyl acylglycerols, based on the total weight of the polar lipid fraction.

In particular embodiments of the above mentioned Emulsifier Extracts of the invention (crude or purified extracts), the polar lipid phase comprises at least 5 wt % of sulphur lipids (such as sulfoquinovosyldiacylglycerol (SQDG), in another embodiment at least 8 wt %, for example at least 10 wt %, at least 11 wt %, at least 12 wt %, at least 20%, or at least 30% of sulphur lipids (such as sulfoquinovosyldiacylglycerol (SQDG) based on the total weight of the oil fraction.

In particular embodiments of the above mentioned Emulsifier Extracts of the invention (crude or purified extracts), the polar lipid phase comprises at least 5 wt % of sulphur lipids (such as sulfoquinovosyldiacylglycerol (SQDG), in another embodiment at least 8 wt %, for example at least 10 wt %, at least 11 wt %, at least 12 wt %, at least 20%, or at least 30% of sulphur lipids (such as sulfoquinovosyldiacylglycerol (SQDG) based on the total weight of the polar lipid fraction.

In particular embodiments of the above mentioned Emulsifier Extracts of the invention (crude or purified extracts), the polar lipid phase comprises at least 5 wt % of phospholipids (such as phosphatidylcholine (PC), phosphatidylethanolamine (PE) or phosphatidylglycerol (PG)) and/or lysophospholipids (the monoacyl forms of PC, PE, or PG), such as at least 8 wt %, at least 10 wt %, at least 11 wt %, at least 12 wt %, at least 20%, or at least 30% of phospholipids (such as phosphatidylcholine (PC), phosphatidylethanolamine (PE) or phosphatidylglycerol (PG)) and/or lysophospholipids (the monoacyl forms of PC, PE, or PG), based on the total weight of the oil fraction.

In particular embodiments, the galactosyl acylglycerols comprised in the polar lipid fraction include at least one of monogalacosyldiacylglycerols and/or diacylgalactosyldiacylglycerols and/or digalactosylmonoacylglycerol and/or monagalactosylmonoacylglycerol.

As already mentioned before, the inventors have surprisingly found that extracts with high emulsification properties can be obtained from a very diverse spectrum of micro and macroalgae, photosynthetic bacteria or photosynthetic organs and tissues of plants, using specific solvents.

The inventors have surprisingly found that the extracts of the invention have very high emulsification properties such as two or three times better than state-of-the-art standard natural emulsifiers (oat oils or oat extracts rich in polar lipids).

Thus, the invention is also related to the use of at least one Extract of the invention (crude extracts of the invention and/or the purified extracts rich in polar lipids of the invention) as emulsifier.

The invention is related to the use of one or more extract(s) obtained or obtainable from photosynthetic parts of plants (such as leaves or stems) and/or one or more purified extract(s) rich in polar lipids obtained or obtainable from photosynthetic parts of plants (such as leaves or stems) as described herein as emulsifiers.

In one embodiment, the photosynthetic parts of said plants may be the leaves and/or the stems.

The invention is related to the use of one or more plant green of the invention obtained or obtainable from greens of plants including but not limited to greens of broccoli rabe, broccoli, red radish, guarana, rosemary, sage, thyme, mint, basil, Perilla frutescens, ajwain, angelica, anise, asafoetida, caraway, carrot, celery, chervil, coriander, cumin, dill, fennel, lovage, cow parsley, parsley, parsnip, sea holly, silphium oregano, lettuce, alfalfa fenugreek (Trigonella foenum-graecum), lentil (Lens culinaris), lupine (genus Lupinus), pea (Pisum sativum), garlic, scallion, leek, chive, chinese onion, onions, green onions, beetroot, spinach, parsley, yerba mate, tea, endive, watercress, nettle, carrots, and mixtures thereof, and optionally the solvent is selected from alcohol or alcohol water mixtures, such as ethanol 100% or ethanol:water (70:30, 80:20 or 90:10), and optionally the lipid contain is from at least 5%, such as least 10% of polar lipids.

In certain embodiments, the extract is an extract obtained or obtainable from the greens of spring onion and is extracted using ethanol 100%.

In certain embodiments, the Extract is an extract obtained or obtainable from alfalfa using ethanol:water as solvent (such as 80:20 or 90:10 ethanol:water). In certain embodiments, the alfalfa extract also comprises saponins. In certain embodiments, the alfalfa extract comprises at least 10% of polar lipids, such as at least 15% w/w, such as 18% and optionally comprises at least 15% w/w of saponins, such as at least 20% of saponins.

The invention is also related to the use as emulsifier of an extract obtained or obtainable from the greens of spring onion (such as a crude and/or a purified extract), optionally that is extracted using ethanol 100%.

The invention is also related to the use as emulsifier of an Extract obtained or obtainable from alfalfa (such as a crude and/or a purified extract), optionally using ethanol 100% or ethanol-water as solvent (such as 80:20 or 90:10 ethanol:water). In certain embodiments, the alfalfa extract also comprises saponins.

The invention is also related to the use as emulsifier of an Extract obtained or obtainable from the greens of broccoli rabe, green pea pods, rosemary, celery, carrot, parsley, radish leaves and/or black tea leaves, optionally that is extracted using ethanol 100% or ethanol-water (such as 90:10 or 80:20) as solvent.

The invention is related to the use of an Extract obtained or obtainable from Spinach (such as a crude Spinach extract and/or a purified Spinach extract) as emulsifier. In certain embodiments, the Spinach extract (crude or purified extract) is obtained from the leaves and/or stems using ethyl acetate, isopropanol, acetone or hydroa-cetone, chloroform:methanol (such as 2:1), methanol, and/or ethanol or hydro-ethanol or mixtures thereof as extraction solvent. In certain embodiments, the spinach extract of the invention (crude or purified) is rich in polar lipids.

The invention is also related to the use of one or more microalgae crude extracts and/or one or more microalgae purified extracts rich in polar lipids as emulsifiers. In one embodiment, the microalgae extract is obtained or obtainable from the microalgae including but not limited to Chlorella (such as Chlorella vulgaris, Chlorella sorokiniana, Chlorella zofingensis), Chysophyceae, Xantophyceae, Baccilariophyceae, Dinophyceae, Rodophyceae, Phaeophyceae, Chlorophyceae, Prasinophyceae, Cryptophyceae, Crypthecodinium, Cylindrothec, Botrycoccus, Dunaliella (such as Dunaliella salina), Euglena gracilis, Isochrysis, Tetraselmis, Nannochloropsis, Neochloris, Nitzschia Scenedesmus, Chlorobotrys, Eustigmatos, Phaeodactylum, Porphyridium, Pseudostaurastrum, Schizochytrium, Tetraselmis, Vischeria, Monodopsis, Ellipsoidion, Pseudocharaciopsis and combinations thereof. In certain embodiments, the microalgae extract of the invention is extracted using a solvent that is selected from ethyl acetate, isopropanol, acetone, chloroform:methanol (such as 2:1), methanol, MeTHF, ethanol and/or hydro-ethanol and mixtures thereof.

In a preferred embodiment, the emulsifier is a Chlorella purified extract rich in polar lipids and/or a Chlorella crude extract. In one embodiment, the Chlorella purified extract rich in polar lipids and/or the crude extract is obtained using methyl tetrahydrofuran, isopropanol, acetone or hydro-acetone, water, ethanol or hydro-ethanol (such as 90:10 ethanol:water) or mixtures thereof as extraction solvent. The Chlorella that may be used in the present invention includes but is not limited to Chlorella vulgaris, Chlorella sorokiniana and Chlorella zofingensis.

The invention is related to the use of an Extract obtained or obtainable from Chlorella (such as a crude Chlorella extract and/or a purified Chlorella extract) as emulsifier. In certain embodiments, the Chlorella extract (crude or purified extract) is obtained using methyl tetrahydrofuran, isopropanol, acetone or hydro-acetone, water, ethanol or hydro-ethanol (such as 90:10 ethanol:water) or mixtures thereof as extraction solvent.

It is also described the use as emulsifier of a Chlorella extract from Chlorella vulgaris, Chlorella zofingensis and/or Chlorella sorokiniana, that is obtained or obtainable using ethanol or ethanol and water (such as 100% ethanol, 90:10, 80:20 or 70:30 EtOH:water) as solvent and, optionally, having a polar lipid contain of at least 40%, such as at least 50%, such as at least 70%, at least 80% or at least 90%.

It is also described the use as emulsifier of a Tetraselmis extract (crude or purified) obtained or obtainable using Ethanol or ethanol and water (such as 100% ethanol, 80:10 or 90:10 EtOH:water) as solvent and, optionally, having a polar lipid contain of at least 50%, such as at least 60%, such as at least 70%, such as at least 80%, such as at least 90%, such as 69%.

It is also described the use as emulsifier of a Nannochloropsis extract (crude or purified) obtained or obtainable using ethanol or ethanol and water (such as 100% ethanol, 90:10, 80:20 or 70:30 EtOH:water) as solvent and, optionally, having a polar lipid contain of at least 20%, of at least 50%, such as at least 60%, such as at least 70%, at least 80% or at least 90%.

It is also described the use as emulsifier of a Nannochloropsis extract (crude or purified) obtained or obtainable using isopropanol, acetone and/or ethylacetate as solvent and, optionally, having a polar lipid contain of at least 30%, such as at least 60%, such as at least 70%, such as at least 80%.

It is also described the use as emulsifier of a Dunaliella salina obtained or obtainable using Ethanol, ethanol and water (such as 100% ethanol, 90:10, 80:20 or 70:30 EtOH:water), isopropanol, acetone, MTHF, water and/or ethylacetate as solvent and, optionally, having a polar lipid contain of at least 20%, such as at least 50%, such as at least 70%, at least 80% or at least 90%.

It is also described the use as emulsifier of a Chlorella extract (crude or purified) such as from Chlorella vulgaris, Chlorella zofingensis and/or Chlorella sorokiniana, that is obtained or obtainable using isopropanol, acetone and/or ethylacetate as solvent and, optionally, having a polar lipid contain of at least 30%, such as at least 60%, such as at least 70%, such as at least 80%.

It is also described the use as emulsifier of a Isochrysis extract (crude or purified) that is obtained or obtainable using ethanol or ethanol and water (such as 100% ethanol, 90:10, 80:20 or 70:30 EtOH:water) as solvent and, optionally, having a polar lipid contain of at least 20%, such as at least 50%, such as at least 70%, at least 80% or at least 90%.

It is also described the use as emulsifier of a Isochrysis extract (crude or purified) that is obtained or obtainable using isopropanol, acetate and/or acetone as solvent and, optionally, having polar lipid contain of at least 20%, such as at least 50%, such as at least 70%, at least 80% or at least 90%.

The invention is also related to the use of one or more macroalgae crude extracts of the invention and/or one or more macroalgae purified extracts rich in polar lipids of the invention as emulsifiers. In one embodiment, the macroalgae are selected from Ascophyllum nodosum, Fucus serratus, F. vesiculosus, Himanthalia elongata, Undaria pinnatifida, Laminaria digitata, L. saccharina, L. japonica, Alaria esculenta, Palmaria palmata (dulse), Porphyra umbilicalis, P. tenera, P. yezoensis, P. dioica, P. purpurea, P. laciniata, P. leucostica, Chondrus crispus, Gracilaria verrucosa, Lithothamnium calcareum, Enteromorpha spp., or Ulva spp. In a preferred embodiment, the emulsifier is Ulva spp and/or Agarophyton chilensis crude extract(s) of the invention and/or Ulva spp and/or Agarophyton chilensis purified extract(s) rich in polar lipids.

It is also described the use as emulsifier of a Ulva spp and/or Agarophyton chilensis crude extract(s) or purified extract(s) rich in polar lipids that is obtained or is obtainable using methyl tetrahydrofuran, isopropanol, ethanol or hydroethanol (such as 90:10 ethanol:water) as extraction solvent or mixtures thereof.

The invention is also related to the use of one or more photosynthetic bacterial crude extracts (or photosynthetic bacterial crude extracts of the invention) and/or one or more photosynthetic bacterial purified extracts rich in polar lipids (or photosynthetic bacterial purified extracts of the invention) as described herein as emulsifiers. Photosynthetic bacteria may be spirulina and/or Aphanizomenon flosaquae.

In a preferred embodiment, the emulsifier is a spirulina crude extract and/or purified extract rich in polar lipids. In one embodiment, the spirulina extract or the spirulina purified extract rich in polar lipids is obtained using methyl tetrahydrofuran, isopropanol, ethanol or hydro ethanol (such as 90:10 ethanol water) or mixtures thereof as extraction solvent.

It is also described the use as emulsifier of a spirulina crude extract and/or a spirulina purified extract rich in polar lipids that is obtained or is obtainable using methyl tetrahydrofuran, acetone, hexan, isopropanol, ethanol 100% and/or hydro-ethanol (such as 60:40, 70:30, 80:20, 90:10 ethanol-water) as extraction solvent, and optionally having at least 5% or polar lipids, such as at least 10%, at least 20%, at least 30%, at least 40%, at least 50%, at least 60%, at least 70%, such as at least 90% of polar lipids.

In particular embodiments of the above mentioned purified extracts of the invention, the polar lipid phase comprises at least 5 wt %, in another embodiment at least 8 wt %, for example at least 10 wt %, at least 11 wt %, at least 12 wt %, at least 15 wt %, at least 20 wt %, at least 25 wt %, at least 30 wt %, at least 40 wt %, at least 50 wt %, at least 60 wt %, at least 70 wt % or at least 80 wt % of galactosyl acylglycerols, based on the total weight of the extract.

In particular embodiments, the galactosyl acylglycerols comprised in the polar oil fraction include at least one of monogalacosyldiacylglycerols and/or diacylgalactosyldiacylglycerols and/or digalactosylmonoacylglycerol and/or monagalactosylmonoacylglycerol.

Conventional emulsifiers include for instance sugar esters, polyglycerol fatty acid esters, polyglycerol polyricinoleate (PGPR), polysorbates (polyoxyethylene sorbitan esters), monoglycerides/diglycerides and their derivatives, sodium stearoyl lactylate (SSL), phospholipids, glycerol monooeleate, amongst others.

Advantageously, the present invention uses one or more crude extract(s) of the invention (such as crude extracts rich in polar lipids) and/or one or more purified extracts of the invention (such as purified extracts rich in polar lipids) obtained or obtainable from photosynthetic parts of plants (such as spinach leaves or stems), macroalgae, microalgae and/or photosynthetic bacteria as described previously to stabilize emulsions. For example, a purified Alfalfa extract of the invention may be combined with a purified spinach extract of the invention to stabilize emulsions.

Thus, the present invention relates to a “emulsifying system” or Emulsifying system of the invention comprising one or more crude extract(s) of the invention (such as crude extracts rich in polar lipids) and/or one or more purified extracts of the invention (such as purified extracts rich in polar lipids) obtained or obtainable from photosynthetic parts of plants (such as spinach or alfalfa leaves or stems Extracts of the invention), macroalgae (such as Ulva spp Extracts of the invention), microalgae (such as Nannochloropsis, Tetraselmis, Isochrysis, Chlorella sorokiniana, Chlorella vulgaris and/or Dunaliella salina Extracts of the invention) and/or photosynthetic bacteria (such as Spirulina) as described previously. Thus, the present invention relates to the use of an “emulsifying system of the invention” for stabilizing emulsions.

In certain aspects the invention is related to an emulsion comprising an “emulsifying system of the invention” as described herein.

The invention also provides an “emulsifying system of the invention” for use as emulsifier in a food or beverage product for humans or animals, a nutritional supplement, a nutraceutical formulation, a fragrance or flavouring, a pharmaceutical or veterinary formulation, an oenological or cosmetic formulation.

In certain aspects the invention is related to an emulsion comprising at least one Extract of the invention such as the crude or purified extracts described previously.

In a preferred embodiment, the emulsion does not need the addition of such conventional emulsifiers or stabilizing agents.

In a preferred embodiment, the extract of the invention (crude or purified) used to stabilize an emulsion comprises polar lipids. In a more preferred embodiment, the extract of the invention is enriched in polar lipids.

All the different embodiments and variations described previously related to the Extracts of the invention apply also for the Emulsions of the invention.

It is also described an Emulsion of the invention comprising at least one Extract of the invention obtained or obtainable from photosynthetic parts of plants (such as spinach and/or alfalfa leaves or stems), macroalgae, microalgae and/or photosynthetic bacteria as described previously.

In a preferred embodiment, the emulsion of the invention is stable at a pH from about 2 to 10, such as 3 to 9, 3 to 8, 3 to 7, 3 to 6, 3 to 5, 4 to 6, 4 to 7 or 5 to 10.

In certain embodiments, the emulsions of the invention is stable at a pH from about 2 to 10, such as 3 to 9, 3 to 8, 3 to 7, 3 to 6, 3 to 5, 4 to 6, 4 to 7 or 5 to 10.

In certain embodiments, the emulsion stabilized with the at least one Extract of the invention or with the “emulsifying system of the invention” is water in oil emulsion or oil in water emulsions.

In the present specification, the term “lipid phase” is understood as including any solid and/or liquid ingredient miscible with oil or fat or that has the capacity to dissolve in oil or fat, and “aqueous phase” as including any solid and/or liquid ingredient soluble or miscible with water or that has the capacity to dissolve in water.

The emulsions stabilized with the crude extracts of the invention and/or with the purified extracts rich in polar lipids according to the invention can be prepared following conventional methods for the preparation of emulsions.

According to an exemplary method, the emulsion of the invention may be prepared by a process comprising:
(a) mixing ingredients of the aqueous phase;
(b) mixing ingredients of the lipid phase;
(c) dispersing the one or more Extracts of the inventions (such as one or more crude extract(s) of the invention and/or one or more purified extract(s) rich in polar lipids according to the invention) in one or both of the aqueous phase or the lipid phase;
(d) homogenizing the two phases to form an emulsion.

According to an exemplary method, in some embodiments, the process for preparing an emulsion or a food product in the form of an emulsion comprises the steps of:
(a) mixing ingredients of the aqueous phase;
(b) mixing ingredients of the lipid phase;
(c) dispersing the one or more Extracts of the inventions (such as one or more crude extract(s) of the invention and/or one or more purified extract(s) rich in polar lipids according to the invention) in one or both of the aqueous phase or the lipid phase;
(d) homogenizing the two phases to form an emulsion.

In another embodiment, the invention is related to an emulsion prepared by a process comprising:
a) mixing ingredients of an aqueous phase;
b) mixing ingredients of a lipid phase;
c) dispersing one or more Extracts of the inventions (such as one or more crude extract(s) of the invention and/or one or more purified extract(s) rich in polar lipids according to the invention) in one or both of the aqueous phase or the lipid phase; and
d) homogenizing the two phases to form an emulsion.

In certain embodiments, for the preparation of an oil-in-water emulsion, the crude extract of the invention and/or the purified extract comprising polar lipids (such as a purified extract rich in polar lipids) according to the invention are dispersed in the aqueous phase, and the oil/fat phase is added to the aqueous phase, before agitation to form an emulsion. In other embodiments, for the preparation of a water-in-oil emulsion, the crude extract of the invention and/or the purified extract comprising polar lipid (such as a purified extract rich in polar lipids) according to the invention are dispersed in the oil/fat phase, and the aqueous phase is added to the oil/fat phase, before agitation to form an emulsion.

Homogenization is conveniently used to provide the agitation for formation of the emulsion; however, other conventional technologies are contemplated such as high shear, colloid mill such as bead or ball mill, high pressure homogenization, mixing vessel equipment, ultrasound, membrane like ultrafiltration or microfiltration, etc.

The inventors have surprisingly found that the emulsions of the invention obtained using the crude extracts and/or the purified extracts rich in polar lipids according to the invention are very stable at a very large range of pH conditions. Thus, in a further embodiment, the crude extracts of the invention and/or the purified extract(s) rich in polar lipids according to the present invention can be used as emulsifiers or emulsifying systems at a pH from about 2 to 10, such as 3 to 9, 3 to 8, 3 to 7, 3 to 6, 3 to 5, 4 to 6, 4 to 7 or 5 to 10.

In certain embodiments, the emulsions of the invention has a pH from about 2 to 10, such as 3 to 9, 3 to 8, 3 to 7, 3 to 6, 3 to 5, 4 to 6, 4 to 7 or 5 to 10.

The inventors have surprisingly found that the emulsions of the invention obtained using the crude extracts of the invention and/or the purified extracts rich in polar lipids according to the invention are very stable over time and have a very small droplet size. Droplet size may be measured as described in the examples of the present application or using any other method known in the art.

In certain embodiments, the droplets size (Dᵥ98) of the emulsion is preferably comprised between 0.05 and 50 μm, more preferably between 0.5 and 10 μm, and even more preferably between 0.5 and 2 μm.

The droplet size remains stable i.e. remains within such range for at least one day of storage at ambient temperature (25° C.). In another embodiment, the droplet size remains within said range for at least 4, at least 5, at least 6, at least 7, at least 8, 9, 10, 15 or at least 20 days of storage at ambient temperature.

In one embodiment, the emulsifier or emulsifying system comprises or consist of one or more spinach Extracts of the invention (such as crude extract(s) or Spinach Purified extracts rich in polar lipids) obtained or obtainable using ethyl acetate, isopropanol, ethanol:water (such as 90:10), ethanol, acetone, methanol, acetone:water (such as 90:10), and/or acetone:water (such as 80:20). In a preferred embodiment, the droplet size of the emulsion is preferably comprised between 0.05 to 50 μm, such as between 0.5 and 2 μm. In one embodiment, the pH of the emulsion obtained is from 2 to 10, such as from 3 to 5 such as 3.5.

In one embodiment, the emulsifier or emulsifying system comprises or consist of one or more spinach crude extract(s) and/or Spinach Purified extracts rich in polar lipids obtained or obtainable using ethanol:water (such as 60:40), methyl tetrahydrofuran, ethanol, ethyl acetate, hexane, isopropanol, methanol, acetone:water (such as 80:20), acetone, ethanol:water (such as 90:10), acetone:water, (such as 90:10), and/or ethanol. In a preferred embodiment, the droplet size of the emulsion obtained is preferably comprised between 0.05 and 50 μm, such as between 0.5 and 2 μm. In one embodiment, the pH of the emulsion is from 2 to 10, such as from 5 to 8 such as 7.

In one embodiment, the emulsifier or emulsifying system comprises or consist of one or more spirulina crude extract(s) and/or spirulina purified extracts rich in polar lipids obtained or obtainable using methyl tetrahydrofuran, ethanol, ethanol:water (such as 90:10) and/or isopropanol. In a preferred embodiment, the droplet size of the emulsion obtained is preferably comprised between 0.05 to 50 μm, such as between 0.5 and 3.5 μm, such as 1.5 μm. In one embodiment, the pH of the emulsion is from 2 to 10, such as from 3 to 5 such as 3.5.

In one embodiment, the emulsifier or emulsifying system comprises or consist of one or more spirulina crude extract(s) and/or spirulina purified extracts rich in polar lipids obtained or obtainable using methyl tetrahydrofuran, ethanol:water (such as 90:10), ethanol and/or isopropanol. In a preferred embodiment, the droplet size of the emulsion obtained is preferably comprised between of 0.05 to 50 μm, such as between 1 and 3.5 μm. In one embodiment, the pH of the emulsion is from 2 to 10, such as from 5 to 8 such as 7.

In one embodiment the emulsifier, the emulsifier system or emulsion of the invention comprises or consist of one or more of spinach extract (such as crude extract and/or purified extract), alfalfa extract (such as crude extract and/or purified extract), greens of spring onion extract (such as crude extract and/or purified extract), Dunaliella salina extract (such as crude extract and/or purified extract), Chlorella vulgaris extract (crude extract and/or purified extract), Chlorella zofingensis extract (such as crude extract and/or purified extract), Chlorella sorokiniana (such as crude extract and/or purified extract), Isochrysis extract (such as crude extract and/or purified extract), Nannochloropsis extract (such as crude extract and/or purified extract), Tetraselmis extract (such as crude extract and/or purified extract), Ulva spp extract (such as crude extract and/or purified extract), optionally rich in polar lipids, that is obtained or obtainable using ethanol 100% and/or ethanol:water (such as 90:10, 80:20 or 70:30 EtOH water). In a preferred embodiment the droplet size of the emulsion obtained is preferably comprised between of 0.05 to 50 μm, such as between 0.5 and 3.5 μm. In one embodiment, the pH of the emulsion is from 2 to 10, such as from 5 to 8, or such as 3 to 5.

In a preferred aspect of the invention, the crude extracts and/or the purified extracts rich in polar lipids or the emulsifying systems of the inventions are used in an amount of from 0.05% to 20% by weight, such as from 0.1 to 10% by weight, more preferably from 0.3 to 5%, from 1% to 3% by weight, relative to the total weight of the emulsion.

The invention also provides one or more crude extract(s) of the invention and/or one or more purified extract(s) rich in polar lipids according to the invention for use as emulsifier in a food or beverage product for humans or animals, a nutritional supplement, a nutraceutical formulation, a fragrance or flavouring, a pharmaceutical or veterinary formulation, an oenological or cosmetic formulation.

Food encompasses the following general food categories, as defined by the Food and Drug Administration (FDA): baked goods and baking mixes, including all ready-to-eat and ready-to-bake products, flours, and mixes requiring preparation before serving; beverages, alcoholic, including malt beverages, wines, distilled liquors, and cocktail mix; beverages and beverage bases, non-alcoholic, including only special or spiced teas, soft drinks, coffee substitutes, and fruit and vegetable flavored gelatin drinks; breakfast cereals, including ready-to-eat and instant and regular hot cereals; cheeses, including curd and whey cheeses, cream, natural, grating, processed, spread, dip, and miscellaneous cheeses; chewing gum, including all forms; coffee and tea, including regular, decaffeinated, and instant types; condiments and relishes, including plain seasoning sauces and spreads, olives, pickles, and relishes, but not spices or herbs; confections and frostings, including candy and flavored frosting, marshmallows, baking chocolate, and brown, lump, rock, maple, powdered, and raw sugars; dairy product analogs, including nondairy milk, frozen or liquid creamers, coffee whiteners, toppings, and other nondairy products; egg products, including liquid, frozen, or dried eggs, and egg dishes made therefrom, i.e., egg roll, egg foo young, egg salad, and frozen multicourse egg meals, but not fresh eggs; fats and oils, including margarine, dressings for salads, butter, salad oils, shortenings and cooking oils; fish products, including all prepared main dishes, salads, appetizers, frozen multicourse meals, and spreads containing fish, shellfish, and other aquatic animals, but not fresh fish; fresh eggs, including cooked eggs and egg dishes made only from fresh shell eggs; fresh fish, including only fresh and frozen fish, shellfish, and other aquatic animals; fresh fruits and fruit juices, including only raw fruits, citrus, melons, and berries, and home-prepared “ades” and punches made therefrom; fresh meats, including only fresh or home-frozen beef or veal, pork, lamb or mutton and home-prepared fresh meat-containing dishes, salads, appetizers, or sandwich spreads made therefrom; fresh poultry, including only fresh or home-frozen poultry and game birds and home-prepared fresh poultry-containing dishes, salads, appetizers, or sandwich spreads made therefrom; fresh vegetables, tomatoes, and potatoes, including only fresh and home-prepared vegetables; frozen dairy desserts and mixes, including ice cream, ice milks, sherbets, and other frozen dairy desserts and specialties; fruit and water ices, including all frozen fruit and water ices; gelatins, puddings, and fillings, including flavored gelatin desserts, puddings, custards, parfaits, pie fillings, and gelatin base salads; grain products and pastas, including macaroni and noodle products, rice dishes, and frozen multicourse meals, without meat or vegetables; gravies and sauces, including all meat sauces and gravies, and tomato, milk, buttery, and specialty sauces; hard candy and cough drops, including all hard type candies; herbs, seeds, spices, seasonings, blends, extracts, and flavorings, including all natural and artificial spices, blends, and flavors; jams and jellies, home-prepared, including only home-prepared jams, jellies, fruit butters, preserves, and sweet spreads; jams and jellies, commercial, including only commercially processed jams, jellies, fruit butters, preserves, and sweet spreads; meat products, including all meats and meat containing dishes, salads, appetizers, frozen multicourse meat meals, and sandwich ingredients prepared by commercial processing or using commercially processed meats with home preparation; milk, whole and skim, including only whole, low fat, and skim fluid milks; milk products, including flavored milks and milk drinks, dry milks, toppings, snack dips, spreads, weight control milk beverages, and other milk origin products; nuts and nut products, including whole or shelled tree nuts, peanuts, coconut, and nut and peanut spreads; plant protein products, including the National Academy of Sciences/National Research Council “reconstituted vegetable protein” category, and meat, poultry, and fish substitutes, analogs, and extender products made from plant proteins; poultry products, including all poultry and poultry-containing dishes, salads, appetizers, frozen multicourse poultry meals, and sandwich ingredients prepared by commercial processing or using commercially processed poultry with home preparation; processed fruits and fruit juices, including all commercially processed fruits, citrus, berries, and mixtures; salads, juices and juice punches, concentrates, dilution, “ades”, and drink substitutes made therefrom; processed vegetables and vegetable juices, including all commercially processed vegetables, vegetable dishes, frozen multicourse vegetable meals, and vegetable juices and blends; snack foods, including chips, pretzels, and other novelty snacks; soft candy, including candy bars, chocolates, fudge, mints, and other chewy or nougat candies; soups, home-prepared, including meat, fish, poultry, vegetable, and combination home-prepared soups; soups and soup mixes, including commercially prepared meat, fish, poultry, vegetable, and combination soups and soup mixes; sugar, white, granulated, including only white granulated sugar; sugar substitutes, including granulated, liquid, and tablet sugar substitutes; and sweet sauces, toppings, and syrups, including chocolate, berry, fruit, corn syrup, and maple sweet sauces and toppings.

In a preferred embodiment, the application of the emulsion of the invention is on sauces, mayonnaises, snacks, ice creams and desserts, dairy products (such as vegetal milks), beverages, sausages and condiments, process products (meat), meat analogues, coffee creamers, baked goods, spreads, or margarines, etc.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

When we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of we mean that the extract or composition being described must contain the listed ingredient(s) only. The term “about” as used herein, e.g. when referring to a measurable value (such as an amount or weight of a particular component in the reaction mixture), refers to variations of ±20%, ±10%, ±5%, ±1%, ±0.5%, or, particularly, ±0.1% of the specified amount.

### FIGURES

FIG. 1. Mass yield (%) obtained for spinach leave extraction using various solvents. Independently duplicated result are indicated with the mention n=2.

FIG. 2. Mass yield (%) obtained for spinach leave extraction using a Soxhlet apparatus and various chloroform:methanol mixtures as extraction solvents.

FIG. 3. Polar lipid content (%) in crude extracts of spinach leaves obtained by a typical S/L extraction procedure or by a Soxhlet procedure (only for chloroform:methanol mixtures).

FIG. 4. Emulsifying activity of the spinach leaves crude extracts (1%) in oil-in-water emulsion at pH 3.5 as measured by the droplet size (Dᵥ98) and compared to the reference extracts (oat). Droplet size values for spinach and oat extracts are expressed as the average of two independent replicates ±the standard deviation.

FIG. 5. Emulsifying activity of the spinach leaves crude extracts (1%) in oil-in-water emulsion at pH 7 as measured by the droplet size (Dᵥ98) and compared to the reference extracts (oat). Droplet size values for spinach and oat extracts are expressed as the average of two independent replicates ±the standard deviation.

FIG. 6. Emulsifying activity of the spinach leaves crude extracts (5%) in water-in-oil emulsions at pH 3.5 or 7 as measured by the droplet size (Dᵥ98).

FIG. 7. Mass yield (%) obtained for spirulina extraction cake extraction using various solvents.

FIG. 8. Mass yield (%) obtained for spirulina extraction using a Soxhlet apparatus and various chloroform:methanol mixtures as extraction solvents.

FIG. 9. Polar lipid content (%) in crude extracts of spinach leaves obtained by a typical S/L extraction procedure or by a Soxhlet procedure (only for chloroform:methanol mixtures).

FIG. 10. Emulsifying activity of the spirulina crude extracts (1%) in oil-in-water emulsion at pH 3.5 as measured by the droplet size (Dᵥ98) and compared to the reference extracts (oat). Droplet size values for spinach and oat extracts are expressed as the average of two independent replicates ±the standard deviation.

FIG. 11. Emulsifying activity of the spirulina crude extracts (1%) in oil-in-water emulsion at pH 7 as measured by the droplet size (Dᵥ98) and compared to the reference extracts (oat). Droplet size values for spinach and oat extracts are expressed as the average of two independent replicates ±the standard deviation.

FIG. 12. Emulsifying activity of the spirulina crude extracts (5%) in oil-in-water emulsion at pH 3.5 as measured by the droplet size (Dᵥ98) and compared to the reference extracts (oat). Droplet size values for spinach and oat extracts are expressed as the average of two independent replicates ±the standard deviation.

FIG. 13. Emulsifying activity of the spirulina crude extracts (5%) in oil-in-water emulsion at pH 7 as measured by the droplet size (Dᵥ98) and compared to the reference extracts (oat). Droplet size values for spinach and oat extracts are expressed as the average of two independent replicates ±the standard deviation.

FIG. 14. Mass yield (%) obtained for Ulva spp. extraction using various solvents.

FIG. 15. Emulsifying activity of an ethanol:water 90:10 Ulva crude extracts (1%) in oil-in-water emulsion at pH 3.5 as measured by the droplet size (Dᵥ98) and compared to the reference extract (oat). Droplet size values for the Ulva extract are expressed as the average of two independent replicates ±the standard deviation.

FIG. 16. Emulsifying activity of an ethanol:water 90:10 Ulva crude extracts (1%) in oil-in-water emulsion at pH 7 as measured by the droplet size (Dᵥ98) and compared to the reference extract (oat). Droplet size values for the Ulva extract are expressed as the average of two independent replicates ±the standard deviation.

FIG. 17. Mass yield (%) obtained for Agarophyton chilensis extraction using various solvents.

FIG. 18. Polar lipid content (%) in crude extracts of Agarophyton chilensis.

FIG. 19. Decrease of AE between emulsions stabilized with oat oil (reference) or spinach extracts depending on the applied purification process. Color was measured in emulsions of type 2 (1% extract, pH 7). Averages and standard deviations were calculated from n=2 repetitions for crude extracts (Y06 and Y13), n=3 for the extracts decolorized with the powdered charcoal (Y09, Y10 and Y15), n=1 for the extract decolorized with the R55S filter sheet (Y37), and n=1 for the extract decolorized with the Filtrox filter sheet (Y41).

FIG. 20. Influence of the purification protocol on the droplet size of oil-in-water emulsions stabilized by 1 and 5% of ethanolic:water (90:10) extracts of spinach (crude or decolorized on charcoal, R55S or Filtrox) at pH 3.5 or 7. The standard deviation was calculated from the ES of different extracts. The number of repetitions (n) was n=2 for the crude extracts (Y13 and Y32), n=3 for the extracts decolorized with the charcoal powder (Y05, Y33 and Y39), n=1 for the extract decolorized with the R55S filter sheet (Y37), and n=1 for the extract decolorized with the Filtrox filter sheet (Y41).

FIG. 21. Influence of the purification protocol on the droplet size of oil-in-water emulsions stabilized by 5% of ethanolic:water (90:10) extracts of spirulina (crude or decolorized on charcoal) at pH 3.5 or 7.

FIG. 22: The Dᵥ98 of emulsions prepared with a commercially available soy lecithin (Topcithin, Cargill) is shown in comparison with three purified plant extracts from Spinach.

### EXAMPLES

### Material and Methods

1. Biological Materials Dried Spinach leaves and alfalfa grass were purchased from Hungarian Food Ingredients Ltd., while extraction cakes of spirulina were obtained from a spirulina purchased from C.B.N Spirulina Bioengineering Co., Ltd and extracted with an aqueous solvent. Rosemary leaves were obtained from Naturex. Dried parsley leaves were purchased from VNK B.V. Biddinghuizen, dried black tea leaves (Lipton yellow) as well as fresh plant materials such as green peas, celery, carrot, broccoli rabe, spring onion and radish were purchased from a local supermarket and the green parts (leaves, pods, etc.) were manually sorted to provide the green samples.

Agarophyton chilensis and Ulva sp. were obtained from Kaiso Spa. Nannochloropsis sp. were obtained from Necton and Monzon Biotech, while Chlorella sorokiniana. Isochrysis and Tetraselmis were purchased from Necton. Dunaliella salina was purchased from Monzon.

### 2. Chemicals

For the extractions, ethanol (further referred sometime to as ‘EtOH’) at 99.9% purity was purchased from Christalco, hexane (C6 alcane>98%; n-hexane>45%) from Azelis, acetone (99.5%) from Univar, methanol (99.9%) from Honeyweel, isopropanol (>98%), ethyl acetate (>99%), and chloroform (>98%, stabilized with 0.6% ethanol) from VWR, 2-methyltetrahydrofuran (>99.5% stabilized with 150-400 ppm BHT) from Sigma-Aldrich.

For purification purpose, powdery supercritical water activated carbon (SCW) was purchased from Chemviron (France), while CarbofilCA and R55S activated carbon filter plates were provided by Filtrox (Switzerland) and 3M (United States), respectively.

For the analytics, digalactosyldiacylglycerol (DGDG, plant) was purchased from Avanti Polar Lipids Inc. (Alabaster, Ala., US). Acetonitrile (ACN), methanol (MeOH) and acetic acid (AA) were obtained from Sigma-Aldrich (Saint-Quentin Fallavier, France). Tetrahydrofuran (THF) was obtained from Biosolve Chime (Dieuze, France). Ultrapure water was obtained from a Milli-Q purification system (Millipore, Billerica, Mass., US).

For the preparation of emulsions, MCT (middle chain triglycerides, Mygliol 812, ex Oleo) was purchased from Oleon MV (Belgium), and deionised water was obtained from Adesco (Spain).

### 3. Solid/Liquid Extractions on Dried Biological Materials to Obtain Crude Extracts

Two hundred grams of dried biological material were mixed with 2 L of solvent (3 L for spinach leaves only) for two hours at reflux and mechanically stirred (175 rpm). The solid/liquid weight ratio was thus 1/10 for oat flour, spirulina extraction cakes, Agarophyton chilensis, Ulva spp., Nannochloropsis, Isochrysis, Tetraselmis, Chlorella vulgaris, Chlorella sorokiniana and Chlorella zofingensis, and 1/15 for spinach. The homogenate was filtered through an AF06 filter plate (retention rate: 15-35 μm) on a Büchner apparatus with a slight suction. Filtration is normally quite rapid and when the residue becomes dry, the solution is let to cool down for one hour approximately. When room temperature is reached, a new filtration is done in some cases with an AF31H filter plate (retention rate: 5-12 μm) on the same system to ensure the resulting extract is devoid of any potential solid particles coming from the biological material or from precipitates forming after the temperature reached 25° C. A rotary evaporator is used to remove the solvent from the extract. The solid extract is then freeze-dried with a dry matter typically >90%. For dry matter determination, the extract is placed in an oven at 125° C. We repeated the freeze-drying step whenever the dry matter was found <90%.

### 4. Direct Solid/Liquid Extractions on Fresh Biological Materials to Obtain Crude Extracts

One litre of ethanol was placed in a necked Erlenmeyer flask and heated to 70° C. One hundred grams of fresh biological material were weighted and properly mixed with a classic cooking blender (Moulinex, France) during a few min. The plant was then added to the hot solvent and magnetically stirred at 450 rpm for 2 hours at reflux. The solid/liquid weight ratio was thus 1/10 for vegetables, based on the fresh weight. The rest of the process is essentially the same as the one described above in section 3. The filtration cake is left under the fume hood for 16 hours. An estimation of the alcoholic degree of the recovered solvent was performed with a hydrometer immersed in 500 mL in a graduated cylinder. The dry matter percentage was determined on the fresh biological material before the extraction, and on the filtration cake to calculate the rate of the released water.

### 5. Indirect Solid/Liquid Extractions on Fresh Biological Materials to Obtain Crude Extracts

One litre of ethanol was placed in a necked Erlenmeyer flask and heated to 70° C. Two hundred and fifty grams of fresh biological material were juiced using a cooking juice-extractor device (Philips, Netherlands) at room temperature for 5 min. The residue was recovered and added to the hot solvent to proceed to the extraction, except for one attempt (extract Z69, fresh spinach leaves) where the juice was clarified through an AF31H filtration on a Buchner apparatus, and the resulting filtration cake was pooled with the recovered residue from the juice-extractor, and pressed together through a 1 μm bag (Filtration Group, The Netherlands) by a hydraulic press (SAM Outillage, France) at 15 bar (10 bar stabilised during 1 min) to remove as much residual juice as possible. The dry cake was then extracted according to the rest of the mixing process. The dry matter percentage was determined on the fresh biological material, before and after the juice extraction, and also on the filtration cake after the extraction, to determine both the loss of water and its release rate.

### 6. Soxhlet Extraction for Total Lipid Determination

Ten grams of biological material were mixed with 450 mL of solvent and extracted for eight hours in a Soxhlet apparatus. A rotary evaporator is used to remove the solvent from the extract. The solid extract is then freeze-dried with a dry matter typically >90%. For dry matter determination, the extract is placed in an oven at 125° C. We repeated the freeze-drying step whenever the dry matter was found <90%.

7. Purification with Powdery Activated Carbon to Obtain Purified Extracts

Right after the filtration step described above in section 3 (Solid/liquid extractions to obtain crude extracts), 10% of activated carbon, relative to the starting biological material amount, were added to the filtrate. The mixture was heated to 50° C. for 30 min under magnetic stirring at 300 rpm. A new filtration was performed through an AF31H filter plate on a Büchner apparatus with a slight suction. The rest of the process is essentially the one described above in section 3.

8. Purification with Activated Carbon Filter Plate to Obtain Purified Extracts

At the filtration step described above in section 3 (Solid/liquid extractions to obtain crude extracts), the conventional cellulosic filter plate was replaced by an activated carbon plate either a CarbofilCA plate (Filtrox, Switzerland) or a R55S plate (3M, United States). The filtration was performed as usual and the rest of the process is essentially the same as the one described above in section 3.

### 9. HPLC-ELSD Method to Measure the Polar Lipid Content

Quantification of the polar lipid was performed by reverse phase high performance liquid chromatography (RP-HPLC) (Agilent Technologies instrument, 1260 Infinity Series) using a 380-ELSD (Agilent Technologies) connected to the instrument. An Agilent Infinity Lab Poroshell 120 EC-C8 column 4 μm 3 mm inner diameter and 150 mm length (Agilent Technologies) was used as stationary phase. Separation of the lipids was carried out using an elution gradient analysis displayed in Table 1 with 0.8 mL/min flow rate. Mobile phase A consisted of a mixture of methanol-water-acid acetic (750:250:4; v/v/v), whereas mobile phase B consisted of mixture of acetonitrile-methanol-THF-acid acetic (500:375:125:4; v/v/v/v). Standard lipid solution of DGDG was dissolved in chloroform:methanol (1.5:1; v/v) prior to injection. The HPLC-ELSD settings were kept constant as follows: 4 μL injection volume, column temperature was maintained at 40° C., the ELSD Evaporator and Nebulizer temperature were set at 40° C. Nitrogen was used as a carrier gas with a gas flow rate at 1.2 SLM. Data rate was 80 Hz, Led intensity 90%, smoothing 3.0 seconds and PMT Gain at 8.0. Chromatograms were analyzed with Agilent OpenLab Rev. C.01.06 software.

TABLE 1
Gradient elution method for quantitative analysis of polar lipids

| | % Eluent A | % Eluent B |
|---|---|---|
| Time | MeOH-H₂O-AA | ACN-MeOH-TFH-AA |
| (min) | (750/250/4) | (500/375/125/4) |
| 0 | 90 | 10 |
| 10 | 60 | 40 |
| 30 | 40 | 60 |
| 45 | 30 | 70 |
| 60 | 10 | 90 |
| 65.1 | 90 | 10 |
| 75 | 90 | 10 |

Standard solutions were injected into the HPLC system prior to each measurement in order to establish the calibration curve in quadratic mode from five levels in the range 10 to 1000 ppm of DGDG. For quantification, all compounds were quantified as DGDG.

Extracts from Agarophyton chilensis, oat, spinach, spirulina, and Ulva were dissolved in chloroform:methanol (1.5:1 v/v) and filtered through a 0.45 μm PTFE filter prior to injection. Concentration of samples were 20 mg/mL for Agarophyton and oat, 10 mg/mL for spinach and 5 mg/mL for spirulina and Ulva.

10. Preparation of the Oil-In-Water Emulsions and Measurement of their Droplet Size

According to the present invention, the solubility of the crude or purified extract in oil and water was determined by adding 1% of extract to water or to a vegetable oil respectively (middle chain triglycerides (MCT) oil fraction, Mygliol 812). Depending on the sample in which the extract dissolved better, the crude or purified extract was classified as “oil soluble” or “water soluble”.

For oil soluble crude or purified extracts, a series of oil-in-water emulsions according to the present invention were obtained by performing, for each of them, the steps of:
(i) Mixing a known amount of MCT with a known amount of oil soluble extract in a glass vessel (see Table 2). (For emulsions of type 5 the oil phase was prepared in excess to ensure accurate weighing of the plant extract. If the extract was not completely soluble and sediment resulted, only the supernatant was used in the next step);
(ii) Adding a known amount of deionized water or deionized water adjusted to pH 3.5 with citric acid (see Table 2), in order to obtain an aqueous phase;
(iii) Emulsifying the oil and the aqueous phases with a Branson Digital Sonifier 450, 102c using a 6.3 mm tip. The tip was immersed into—and positioned in the upper third of—the mixture, which was then emulsified for 3 min at 80% amplitude. The emulsification time was split over 5:50 min alternating 10 s pulses and 10 s pauses. The glass vessel was immersed into cold water (10° C.) to cool the emulsion.

For water-soluble crude or purified extracts, a series of oil-in-water emulsions according to the present invention were obtained by performing, for each of them, the steps of:
(i) Mixing a known amount of deionized water or deionized water adjusted to pH 3.5 with citric acid and a known amount of water-soluble extract in a glass vessel (see Table 2) in order to obtain an aqueous phase. (For emulsions of type 5, the aqueous phase was prepared in excess to ensure accurate weighing of the plant extract. If the extract was not completely soluble and sediment resulted, only the supernatant was used in the next step, adding a known amount of MCT (see Table 2).
(ii) Emulsifying the oil and the aqueous phases with a Branson Digital Sonifier 450, 102c (for Alfalfa a SONICS 500 Watt Ultrasonic Processors—VCX was used) using a 6.3 mm tip. The tip was immersed into—and positioned in the upper third of—the mixture, which was then emulsified for 3 min at 80% amplitude (75% for Alfalfa). The emulsification time was split over 5:50 min alternating 10 s pulses and 10 s pauses. The glass vessel was immersed into cold water (10° C.) to cool the emulsion.

All emulsions following the formulas shown in Table 2 were of oil-in-water type.

TABLE 2
Oil-in-water emulsion formulas. The batch size was 7 g for
all plant extracts except for alfalfa where it was 20 g.

| | Emulsion 1 | Emulsion 2 | Emulsion 3 | Emulsion 4 | Emulsion 5 |
|---|---|---|---|---|---|
| Ingredients | (%) | (%) | (%) | (%) | (%) |
| Deionized water | — | 89 | — | 85 | 89.9 |
| Deionized water adjusted | 89 | — | 85 | — | |
| to pH 3.5 with citric acid | | | | | |
| MCT oil | 10 | 10 | 10 | 10 | 10 |
| Crude or purified extract | 1 | 1 | 5 | 5 | 0.1 |

The droplet size distribution of the oil droplets was measured by Static Light Scattering with a Malvern Mastersizer 3000 using laser diffraction particle size analysis and the Mie scattering theory. For the continuous phase, the refractive index of water, and for the dispersed phase, the refractive index of MCT oil, were used respectively.

The measurement cell was filled with degassed deionized water. The emulsions were diluted by adding the emulsion dropwise to the measurement cell following the obscuration measurement in the device. The Dᵥ98 of the droplet size distribution were then calculated using the software implemented in the measurement instrument. The Dᵥ98, is defined as the diameter where 98% of the population (in volume) lies below this value. All measurements were performed at room temperature. All emulsions were stored at 5° C. for 7 days and the droplet size distribution was measured after 1 day and after 7 days to check the stability of the sample.

11. Preparation of the Water-In-Oil Emulsions and Measurement of their Droplet Size

For oil soluble purified extracts, a series of water-in-oil emulsions according to the present invention were obtained by performing, for each of them, the steps of:
(i) Mixing a known amount of MCT with a known amount of oil soluble extract in a glass vessel (Table 3).
(ii) Adding a known amount of deionized water or deionized water adjusted to pH 3.5 with citric acid (see Table 3), to obtain an aqueous phase;
(iii) Emulsifying the oil and the aqueous phases with a Branson Digital Sonifier 450, 102c using a 6.3 mm tip. The tip was immersed into the sample and positioned in its upper third. The mixture was emulsified for 3 min at 80% amplitude. The emulsification time was split over 5:50 min alternating 10 s pulses and 10 s pauses. The glass vessel was immersed into cold water (10° C.) to cool the emulsion.

For water-soluble purified extracts, a series of water-in-oil emulsions according to the present invention were obtained by performing, for each of them, the steps of:
(i) Mixing a known amount of deionized water or deionized water adjusted to pH 3.5 with citric acid and a known amount of water soluble plant extract in a glass vessel (see Table 3) to obtain a polar phase;
(ii) Adding a known amount of MCT (see Table 3).
(iii) Emulsifying the oil and the aqueous phases with a Branson Digital Sonifier 450, 102c using a 6.3 mm tip. The tip was immersed into the sample and positioned in its upper third. The mixture was emulsified for 3 min at 80% amplitude. The emulsification time was split over 5:50 min alternating 10 s pulses and 10 s pauses. The glass vessel was immersed into cold water (10° C.) to cool the emulsion.

All emulsions following the formulas shown in Table 3 were of water-in-oil type.

TABLE 3
Water-in-oil emulsion formulas. The batch size was 30 g.

| | Emulsion 6 |
|---|---|
| Ingredients | (%) |
| Deionized water | 10 |
| MCT oil | 89 |
| purified extract or emulsifier | 1 |

The droplet size distribution of the water droplets was measured by Static Light Scattering with Malvern Mastersizer 3000 using laser diffraction particle size analysis and the Mie scattering theory. For the continuous phase, the refractive index of MCT, and for the dispersed phase, the refractive index of water, were used respectively.

If unpurified samples with high chlorophyll concentrations were used it was not possible to get reliable measurements as the green colour is changing the adsorption index of the continuous phase which is falsifying the measurement. As this could not be adjusted via the measurement parameters only purified extracts were used for water-in-oil emulsions.

The measurement cell was filled with MCT oil. The emulsions were diluted by adding the emulsion dropwise to the measurement cell following the obscuration measurement in the device. The Dᵥ98 of the droplet size distribution were then calculated using the software implemented in the measurement instrument. The Dᵥ98, is defined as the diameter where 98% of the population (Volume) lies below this value. All measurements were performed at room temperature and all emulsions were stored at 5° C. for 1 day and then the droplet size distribution was measured again to check the stability of the sample.

### 12. Calculation of the Emulsifying Score (ES)

We captured the droplet size as well as the stability of the emulsions in one single index referred to as ‘emulsifying score’ or ES.

We first assigned each dataset (droplet size day 0, day 1 and day 7) a ‘stability index’ (SI) according to the rules described in Table 4.

TABLE 4
Algorithmic rules used for the calculation of the stability index (SI)

| Stability | |
|---|---|
| index | Conditions |
| 3 | the increase of the droplet size after one day is larger |
| | than 100% or the droplet size at day 0 is higher than 40 |
| | μm |
| 2.5 | the increase of the droplet size after one day is between |
| | 30 and 100% |
| 2 | the increase of the droplet size after one day is smaller |
| | than 30% and the increase of the droplet size between |
| | day one and day seven is more than 100% |
| 1.5 | the increase of the droplet size after one day is smaller |
| | than 30% and the increase of the droplet size between |
| | day one and day seven is between 30 and100% |
| 1 | the increase of the droplet size after one day is smaller |
| | than 30% and the increase of the droplet size between |
| | day one and day seven is smaller than 30% |

The ES was then calculated according to the following formula:

ES=10*log(SI³*D(98))

The Dᵥ98 is expressed in μm. The lower the ES, the better the emulsifier. Negative ES can even be obtained.

### Example 1. Production of Crude Extracts from Dried Spinach Leaves by S/L Extraction

Two hundred grams of dried flakes of spinach leaves were mixed with 3 L of solvent (ethanol; ethanol:water, 90:10; ethanol:water, 80:20; ethanol:water, 70:30; ethanol:water, 60:40; ethanol:water, 50:50; acetone; acetone:water, 90:10; acetone:water, 80:20; acetone:water, 70:30; hexane, ethyl acetate, isopropanol, methyltetrahydrofuran, and methanol) for two hours at reflux and mechanically stirred (175 rpm). The solid/liquid weight ratio was thus 1/15. The extraction medium was then filtered through an AF06 filter plate (retention rate: 15-35 μm) on a Buchner apparatus with a slight suction to remove the plant residues. A rotary evaporator was used to remove the solvent from the extract by evaporation at reduced pressure. The solid extract was then freeze-dried with a dry matter typically reaching >90%. For dry matter determination, the extract was placed in an oven at 125° C. We repeated the freeze-drying step whenever the dry matter was found <90% until it overpassed this threshold.

FIG. 1 shows the mass yield obtained for the different tested solvents with maximal values for ethanol:water, 80:20 (21.2%), methanol (18.1%), and ethanol:water, 90:10 (17.0%). The minimal yield were obtained with the apolar solvent hexane (1.4%).

### Example 2. Production of Crude Extracts from Dried Spinach Leaves by Soxhlet Extraction Using Chloroform:Methanol Mixtures as Extraction Solvents

Around 6 to 7 grams of dried flakes of spinach leaves were mixed with 450 mL of solvent and extracted for eight hours in a Soxhlet apparatus using chloroform:methanol mixtures as extraction solvents at different volume ratio (2:1, 1:1, or 1:2). The solid/liquid weight ratio was thus ranging from 1/74 to 1/65 depending on the initial amount of spinach. The extraction medium was then filtered through an AF06 filter plate (retention rate: 15-35 μm) on a Buchner apparatus with a slight suction to remove the plant residues. A rotary evaporator was used to remove the solvent from the extract by evaporation at reduced pressure. The solid extract was then freeze-dried with a dry matter typically reaching >90%. For dry matter determination, the extract was placed in an oven at 125° C. We repeated the freeze-drying step whenever the dry matter was found <90%, until it overpassed this threshold.

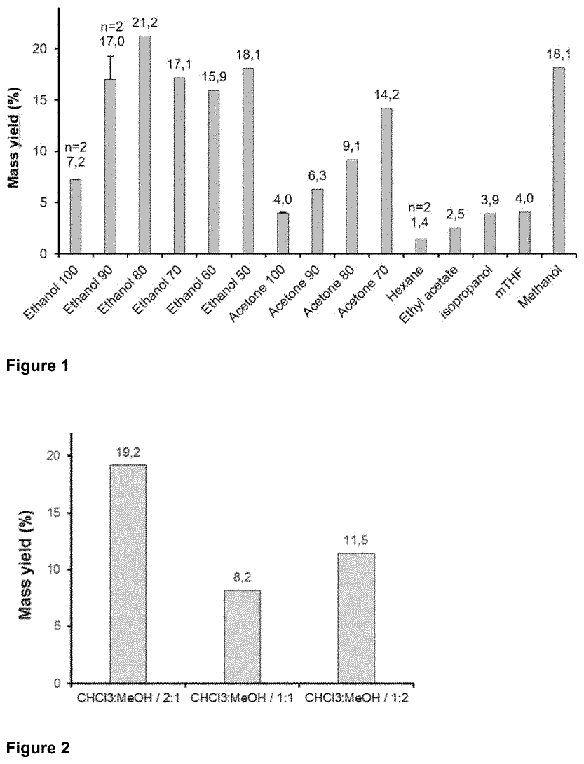

FIG. 2 shows the mass yield obtained for the different tested solvents with maximal values for chloroform:methanol 2:1 (19.2%).

### Example 3. Polar Lipid Characterization of Crude Extracts of Spinach Leaves Obtained by a Typical S/L Extraction Procedure or by a Soxhlet Procedure (Only for Chloroform:Methanol Mixtures)

Quantification of the polar lipid was performed by reverse phase HPLC as described above in section 9 (HPLC-ELSD method to measure the polar lipid content). Results show that the highest polar lipid content (>50%) was reached for crude spinach extracts obtained using ethyl acetate, acetone, chloroform:methanol 2:1 (Soxhlet procedure), and ethanol as extraction solvents (FIG. 3). By contrast, crude extract obtained using ethanol:water mixtures with at least 40% of water resulted in low amounts of polar lipids (<10%).

### Example 4. The Use of 1% of Crude Extracts of Spinach Leaves as Emulsifiers in a 10% Oil-In-Water Emulsion with a Continuous Phase at pH 3.5

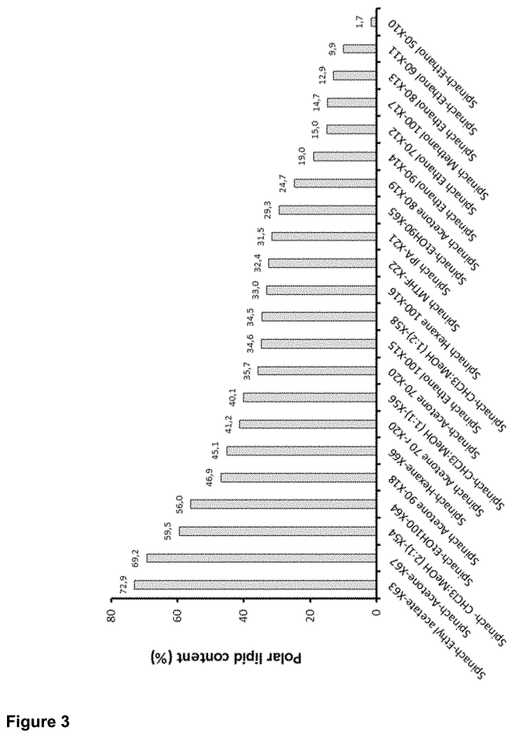

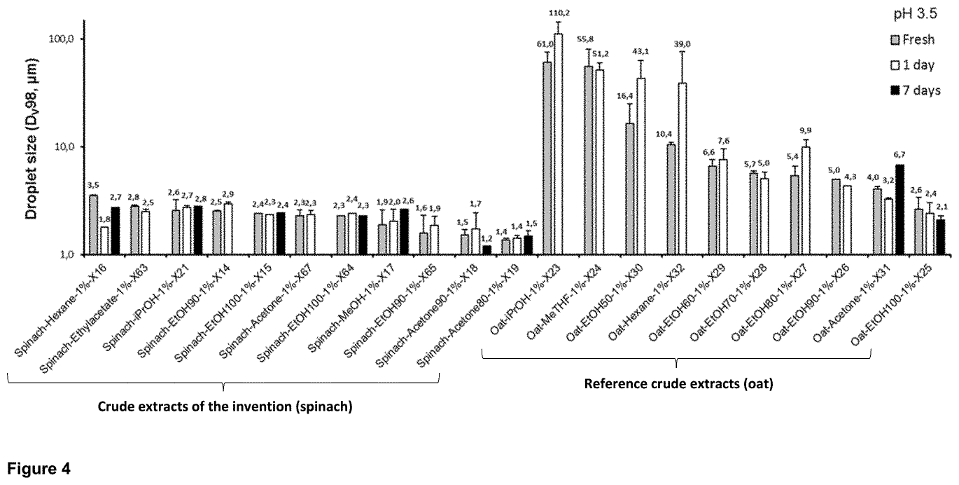

One percent of the different spinach leave crude extracts was used to stabilize oil-in-water emulsions at pH 3.5 made with medium chain triglycerides as the oily phase. The emulsifying activity of these extracts was estimated by the droplet size (Dᵥ98) of the fresh emulsions and its variation over a day of storage (FIG. 4). The variation over a longer storage period (7 days) was also measured for a few selected extracts. Most of the droplet size values are expressed as the average of two independent replicates ±the standard deviation. The lower the droplet size, the higher the emulsifying activity. The higher the increase of the droplet size with time, the lower the stability.

The emulsifying activities of the extracts of the invention (crude spinach extracts) and the stabilities of the emulsions they formed were further compared with those of reference extracts obtained from dehulled oat kernels, a known source of emulsifiers. The extracts of the invention and the reference extracts were obtained using the same conditions of extraction and their emulsifying activities were measured with the exact same protocol. Interestingly, many spinach extracts exhibited a significant ability to form emulsions with smaller droplet sizes than the ones obtained for the reference oat extracts (FIG. 4). Such is the case for the crude spinach extracts obtained using ethyl acetate (Dᵥ98 fresh=2.8 μm), isopropanol (2.6 μm), ethanol:water 90:10 (2.5 and 1.6 μm; two independent extracts referred to as X14 and X65), ethanol (2.4 and 2.3 μm; two independent extracts), acetone (2.3 μm), methanol (1.9 μm), acetone:water 90:10 (1.5 μm), and acetone:water 80:20 (1.4 μm).

### Example 5. The Use of 1% of Crude Extracts of Spinach Leaves as Emulsifiers in a 10% Oil-In-Water Emulsion with a Continuous Phase at pH 7

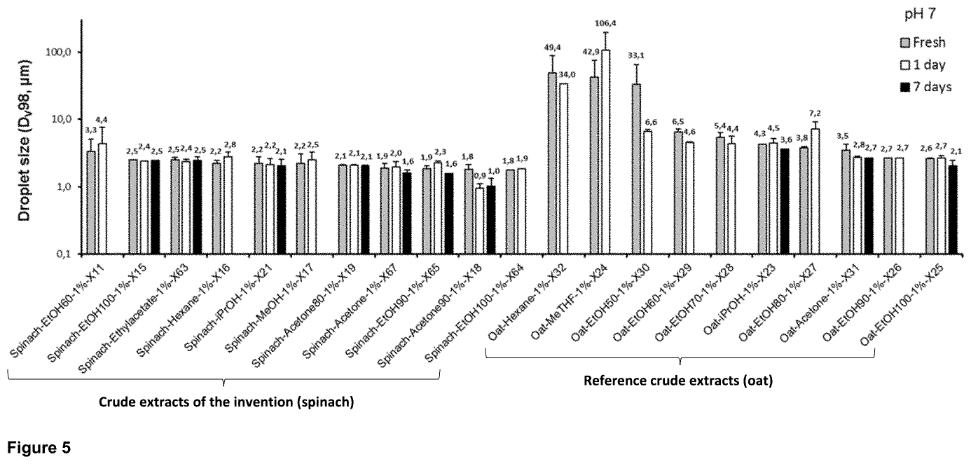

One percent of the different spinach leave crude extracts was used to stabilize oil-in-water emulsions at pH 7 made with medium chain triglycerides as the oily phase. The emulsifying activity of these extracts was estimated by the droplet size (Dᵥ98) of the fresh emulsions and its variation over a day of storage (FIG. 5). The variation over a longer storage period (7 days) was also measured for a few selected extracts. Most of the droplet size values are expressed as the average of two independent replicates ±the standard deviation. The lower the droplet size, the higher the emulsifying activity. The higher the increase of the droplet size with time, the lower the stability.

The emulsifying activities of the extracts of the invention (crude spinach extracts) and the stabilities of the emulsions they formed were further compared with those of reference extracts obtained from dehulled oat kernels, a known source of emulsifiers. The extracts of the invention and the reference extracts were obtained using the same conditions of extraction and their emulsifying activities were measured with the exact same protocol. Interestingly, many spinach extracts exhibited a significant ability to form emulsions with smaller droplet sizes than the ones obtained for the reference oat extracts (FIG. 5). Such is the case for the crude spinach extracts obtained using ethanol:water 60:40 (Dᵥ98 fresh=3.3 μm), methyl tetrahydrofuran (2.6 μm), ethanol (1.8 and 2.5 μm; two independent extracts referred to as X15 and X64), ethyl acetate (2.5 μm), hexane (2.2 μm), isopropanol (2.2 μm), methanol (2.2 μm), acetone:water 80:20 (2.1 μm), acetone (1.9 μm), ethanol:water 90:10 (1.9 μm), acetone:water, 90:10 (1.8 μm), and ethanol (1.8 μm). Among them, the stability of the crude spinach extracts obtained using ethanol:water 60:40 and methyl tetrahydrofuran was not satisfying after one and seven days, respectively.

### Example 6. The Use of 5% of Crude Extracts of Spinach Leaves as Emulsifiers in a Water-In-Oil Emulsion with a Continuous Phase at pH 3.5 or 7

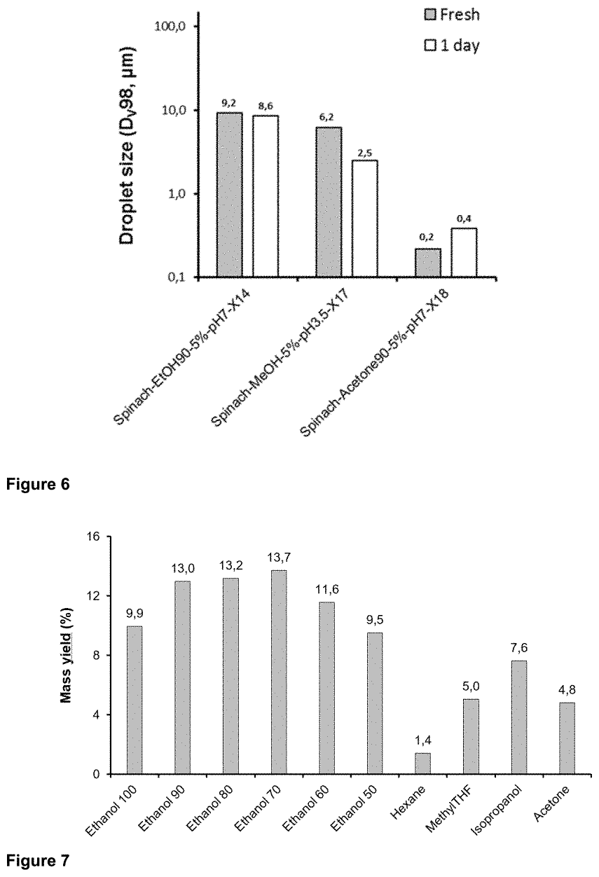

Five percents of spinach leave crude extracts obtained using ethanol:water 90:10, acetone:water 90:10, and methanol, were used to stabilize water-in-oil emulsions at pH 3.5 and 7 made with medium chain triglycerides as the oily phase. The emulsifying activity of these extracts was estimated by the droplet size (Dᵥ98) of the fresh emulsions and its variation over a day of storage (FIG. 6). The lower the droplet size, the higher the emulsifying activity. The higher the increase of the droplet size with time, the lower the stability.

The emulsifying activities of the extracts of the invention (crude spinach extracts) and the stabilities of the emulsions they formed were measured. Interestingly, the tested spinach extracts exhibited a significant ability to form reverse emulsions with relatively small droplet sizes for this type of emulsions (FIG. 6). The obtained Dᵥ98 values for the extract obtained using ethanol:water (90:10) were 9.2 vs. 8.6 μm, for the fresh emulsion and after one day of storage, respectively. They were 6.2 vs. 2.5 μm for the extract obtained using methanol, and 0.2 vs. 0.4 μm for the extract obtained using acetone:water 90:10.

### Example 7. Production of Crude Extracts from Dried Spirulina Extraction Cakes by Sa Extraction

Two hundred grams of dried spirulina (cyanobacteria) extraction cakes were mixed with 2 L of solvent (ethanol; ethanol:water, 90:10; ethanol:water, 80:20; ethanol:water, 70:30; ethanol:water, 60:40; ethanol:water, 50:50; acetone; hexane, isopropanol, and methyltetrahydrofuran) for two hours at reflux and mechanically stirred (175 rpm). The solid/liquid weight ratio was thus 1/10. The extraction medium was then filtered through an AF06 filter plate (retention rate: 15-35 μm) on a Buchner apparatus with a slight suction to remove the plant residues. Filtration is normally quite rapid and when the residue becomes dry, the solution is let to cool down for one hour approximately. When room temperature is reached, a second filtration was done with an AF31H filter paper (retention rate: 5-12 μm) on the same system to ensure the resulting extract was devoid of any potential solid particles coming from the biological material. A rotary evaporator was then used to remove the solvent from the extract by evaporation at reduced pressure. The solid extract was freeze-dried with a dry matter typically reaching >90%. For dry matter determination, the extract was placed in an oven at 125° C. We repeated the freeze-drying step whenever the dry matter was found <90% until it overpassed this threshold.

FIG. 7 shows the mass yield obtained for the different tested solvents with maximal values for ethanol:water, 70:30 (13.7%), ethanol:water, 80:20 (13.2%), and ethanol:water, 90:10 (13.0%). The minimal yield were obtained with the apolar solvent hexane (1.4%).

### Example 8. Production of Crude Extracts from Dried Spirulina Extraction Cakes by Soxhlet Extraction Using Chloroform:Methanol Mixtures as Extraction Solvents

Around 12 to 13 grams of a dried powder of spirulina (cyanobacteria) extraction cakes were mixed with 450 mL of solvent and extracted for eight hours in a Soxhlet apparatus using chloroform:methanol mixtures as extraction solvents at different volume ratios (2:1, 1:1, or 1:2). The solid/liquid weight ratio was thus ranging from 1/35 to 1/38 depending on the initial amount of spirulina. The extraction medium was then filtered through an AF06 filter plate (retention rate: 15-35 μm) on a Buchner apparatus with a slight suction to remove the cyanobacteria residues. Filtration is normally quite rapid and when the residue becomes dry, the solution is let to cool down for one hour approximately. When room temperature is reached, a second filtration was done with an AF31H filter paper (retention rate: 5-12 μm) on the same system to ensure the resulting extract was devoid of any potential solid particles coming from the biological material. A rotary evaporator was used to remove the solvent from the extract by evaporation at reduced pressure. The solid extract was then freeze-dried with a dry matter typically reaching >90%. For dry matter determination, the extract was placed in an oven at 125° C. We repeated the freeze-drying step whenever the dry matter was found <90%, until it overpassed this threshold.

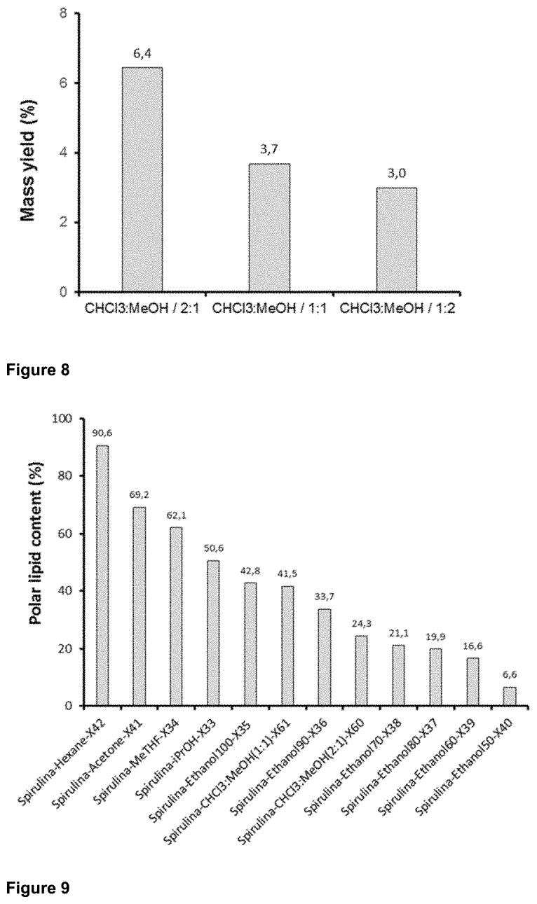

FIG. 8 shows the mass yield obtained for the different tested solvents with maximal values for chloroform:methanol 2:1 (6.4%).

### Example 9. Polar Lipid Characterization of Crude Extracts of Spirulina Extraction Cakes Obtained by a Typical S/L Extraction Procedure or by a Soxhlet Procedure (Only for Chloroform:Methanol Mixtures)

Quantification of the polar lipid was performed by reverse phase HPLC as described above in section 9 (HPLC-ELSD method to measure the polar lipid content). Results show that the highest polar lipid content (>50%) was reached for crude spirulina extraction cake extracts obtained using hexane, acetone, methyltetrahydrofuran (MeTHF), and isopropanol (iPrOH) as extraction solvents (FIG. 9). By contrast, crude extract obtained using ethanol:water mixtures with 50% of water resulted in low amounts of polar lipids (<10%).

### Example 10. The Use of 1% of Crude Extracts of Spirulina Extraction Cakes as Emulsifiers in a 10% Oil-In-Water Emulsion with a Continuous Phase at pH 3.5

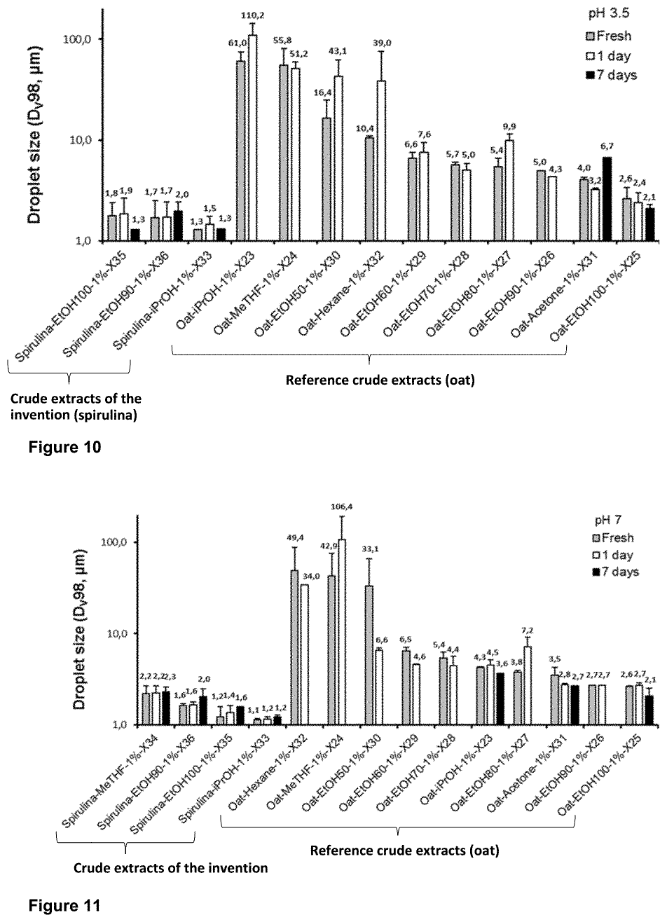

One percent of the different spirulina crude extracts was used to stabilize oil-in-water emulsions at pH 3.5 made with medium chain triglycerides as the oily phase. The emulsifying activity of these extracts was estimated by the droplet size (Dᵥ98) of the fresh emulsions and its variation over a day of storage (FIG. 10). The variation over a longer storage period (7 days) was also measured for selected extracts. Most of the droplet size values are expressed as the average of two independent replicates ±the standard deviation. The lower the droplet size, the higher the emulsifying activity. The higher the increase of the droplet size with time, the lower the stability.

The emulsifying activities of the extracts of the invention (crude spirulina extracts) and the stabilities of the emulsions they formed were further compared with those of reference extracts obtained from dehulled oat kernels, a known source of emulsifiers. The extracts of the invention and the reference extracts were obtained using the same conditions of extraction and their emulsifying activities were measured with the exact same protocol. Interestingly, two spirulina extracts exhibited a significant ability to form emulsions with smaller droplet sizes than the ones obtained for the reference oat extracts (FIG. 10). Such is the case for the crude spirulina extracts obtained using ethanol (Dᵥ98 fresh=1.8 μm) and ethanol:water 90:10 (1.7 μm). Both extracts formed satisfactorily stable emulsions for 7 days.

### Example 11. The Use of 1% of Crude Extracts of Spirulina Extraction Cakes as Emulsifiers in a 10% Oil-In-Water Emulsion with a Continuous Phase at pH 7

One percent of the different crude extracts of spirulina extraction cakes was used to stabilize oil-in-water emulsions at pH 7 made with medium chain triglycerides as the oily phase. The emulsifying activity of these extracts was estimated by the droplet size (Dᵥ98) of the fresh emulsions and its variation over a day of storage (FIG. 11). The variation over a longer storage period (7 days) was also measured for selected extracts. Most of the droplet size values are expressed as the average of two independent replicates ±the standard deviation. The lower the droplet size, the higher the emulsifying activity. The higher the increase of the droplet size with time, the lower the stability.

The emulsifying activities of the extracts of the invention (crude spirulina extracts) and the stabilities of the emulsions they formed were further compared with those of reference extracts obtained from dehulled oat kernels, a known source of emulsifiers. The extracts of the invention and the reference extracts were obtained using the same conditions of extraction and their emulsifying activities were measured with the exact same protocol. Interestingly, several spirulina extracts exhibited a significant ability to form emulsions with smaller droplet sizes than the ones obtained for the reference oat extracts (FIG. 11). Such is the case for the crude spirulina extracts obtained using methyl tetrahydrofuran (Dᵥ98 fresh=2.2 μm), ethanol:water 90:10 (1.6 μm), ethanol (1.2 μm) and isopropanol (1.1 μm).

### Example 12. The Use of 5% of Crude Extracts of Spirulina Extraction Cakes as Emulsifiers in a 10% Oil-In-Water Emulsion with a Continuous Phase at pH 3.5

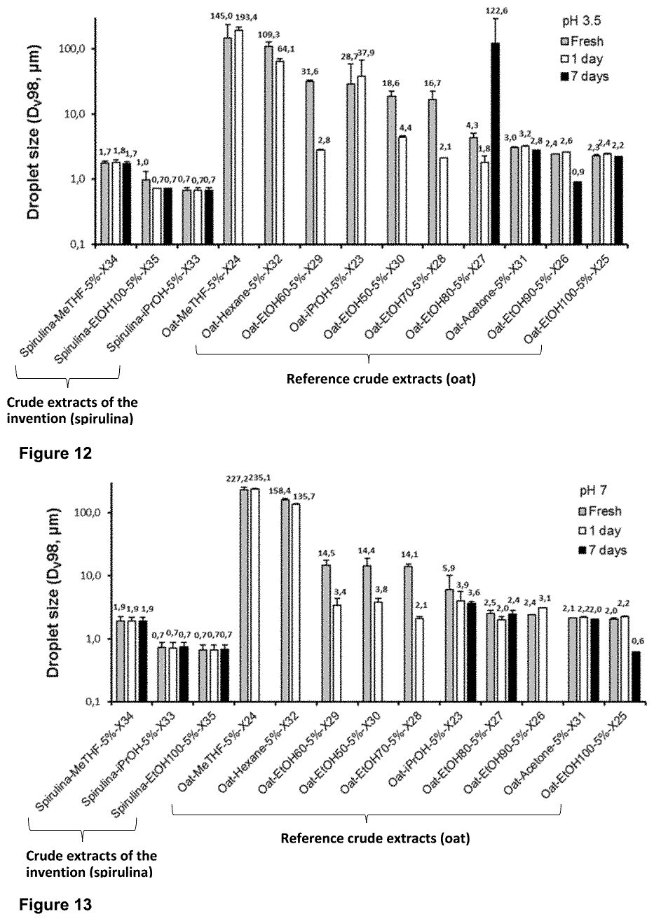

Five percents of the different crude extracts of spirulina extraction cakes were used to stabilize oil-in-water emulsions at pH 3.5 made with medium chain triglycerides as the oily phase. The emulsifying activity of these extracts was estimated by the droplet size (Dᵥ98) of the fresh emulsions and its variation over a day of storage (FIG. 12). The variation over a longer storage period (7 days) was also measured for selected extracts. Most of the droplet size values are expressed as the average of two independent replicates ±the standard deviation. The lower the droplet size, the higher the emulsifying activity. The higher the increase of the droplet size with time, the lower the stability. The emulsifying activities of the extracts of the invention (crude spirulina extracts) and the stabilities of the emulsions they formed were further compared with those of reference extracts obtained from dehulled oat kernels, a known source of emulsifiers. The extracts of the invention and the reference extracts were obtained using the same conditions of extraction and their emulsifying activities were measured with the exact same protocol. Interestingly, several spirulina extracts exhibited a significant ability to form emulsions with smaller droplet sizes than the ones obtained for the reference oat extracts (FIG. 12). Such is the case for the crude spirulina extracts obtained using methyl tetrahydrofuran (Dᵥ98 fresh=1.7 μm), ethanol:water 90:10 (1.6 μm), ethanol (1.0 μm) and isopropanol (0.7 μm). All of them formed satisfactorily stable emulsions for 7 days.

### Example 13. The Use of 5% of Crude Extracts of Spirulina Extraction Cakes as Emulsifiers in a 10% Oil-In-Water Emulsion with a Continuous Phase at pH 7

Five percents of the different crude extracts of spirulina extraction cakes were used to stabilize oil-in-water emulsions at pH 7 made with medium chain triglycerides as the oily phase. The emulsifying activity of these extracts was estimated by the droplet size (Dᵥ98) of the fresh emulsions and its variation over a day of storage (FIG. 13). The variation over a longer storage period (7 days) was also measured for selected extracts. Most of the droplet size values are expressed as the average of two independent replicates ±the standard deviation. The lower the droplet size, the higher the emulsifying activity. The higher the increase of the droplet size with time, the lower the stability.

The emulsifying activities of the extracts of the invention (crude spirulina extracts) and the stabilities of the emulsions they formed were further compared with those of reference extracts obtained from dehulled oat kernels, a known source of emulsifiers. The extracts of the invention and the reference extracts were obtained using the same conditions of extraction and their emulsifying activities were measured with the exact same protocol. Interestingly, several spirulina extracts exhibited a significant ability to form emulsions with smaller droplet sizes than the ones obtained for the reference oat extracts (FIG. 13). Such is the case for the crude spirulina extracts obtained using methyl tetrahydrofuran (Dᵥ98 fresh=1.9 μm), isopropanol (0.7 μm), and ethanol (0.7 μm). All of them formed satisfactorily stable emulsions for 7 days.

### Example 14. Production of Crude Extracts from Dried Ulva Spp. Seaweed (Macroalgae) by S/L Extraction

Two hundred grams of dried Ulva spp. (sea lettuce) were mixed with 2 L of solvent (ethanol, ethanol:water, 90:10; isopropanol, acetone, and methyltetrahydrofuran) for two hours at reflux and mechanically stirred (175 rpm). The solid/liquid weight ratio was thus 1/10. The extraction medium was then filtered through an AF06 filter plate (retention rate: 15-35 μm) on a Buchner apparatus with a slight suction to remove the seaweed residues. When the residue becomes dry, the solution was let to cool down for one hour approximately. When room temperature was reached, a second filtration was done with an AF31H filter paper (retention rate: 5-12 μm) on the same system to ensure the resulting extract was devoid of any potential solid particles coming from the biological material. A rotary evaporator was then used to remove the solvent from the extract by evaporation at reduced pressure. The solid extract was freeze-dried with a dry matter typically reaching >90%. For dry matter determination, the extract was placed in an oven at 125° C. We repeated the freeze-drying step whenever the dry matter was found <90% until it overpassed this threshold.

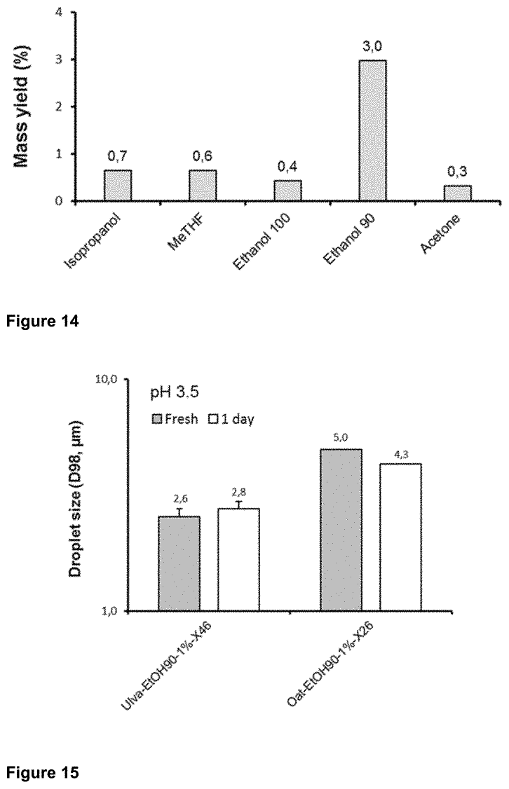

FIG. 14 shows the mass yield obtained for the different tested solvents with maximal values for ethanol:water, 90:10 (3.0%). The minimal yield were obtained with acetone (0.3%) and ethanol (0.4%).

### Example 15. Polar Lipid Characterization of Crude Extracts of Ulva Spp. Seaweed (Macroalgae)

Quantification of the polar lipid was performed by reverse phase HPLC as described above in section 9 (HPLC-ELSD method to measure the polar lipid content). Polar lipid contents of 33.9 and 45.5% were reached for crude Ulva sp. extracts obtained using methyl tetrahydrofuran and ethanol:water, 90:10 (respectively) as extraction solvent.

### Example 16. The Use of 1% of Crude Extracts of Ulva Spp. Seaweed (Macroalgae) as Emulsifiers in a 10% Oil-In-Water Emulsion with a Continuous Phase at pH 3.5

One percent of an Ulva spp. (sea lettuce) crude extract was used to stabilize oil-in-water emulsions at pH 3.5 made with medium chain triglycerides as the oily phase. The emulsifying activity of this extract was estimated by the droplet size (Dᵥ98) of a fresh emulsion and its variation over a day of storage (FIG. 15). The droplet size values for the Ulva spp. extract are expressed as the average of two independent replicates ±the standard deviation. The lower the droplet size, the higher the emulsifying activity. Moreover, the higher the increase of the droplet size with time, the lower the stability.

The emulsifying activity of the extract of the invention (Ulva spp.) and the stability of the emulsion they formed were further compared with those of a reference extract obtained from dehulled oat kernels, a known source of emulsifiers. The extract of the invention and the reference extract were obtained using the same solvent (ethanol:water, 90:10) and conditions of extraction and their emulsifying activities were measured with the exact same protocol. Interestingly, the Ulva spp. extract exhibited a significant ability to form emulsions with smaller droplet sizes (Dᵥ98 fresh=2.6 μm) than the ones obtained for the reference oat extract (5.0 μm) (FIG. 15). Furthermore, the Ulva extract-stabilized emulsion was physically stable after one day of storage at room temperature (2.6 vs. 2.8 μm).

### Example 17. The Use of 1% of Crude Extracts of Ulva Spp. Seaweed (Macroalgae) as Emulsifiers in a 10% Oil-In-Water Emulsion with a Continuous Phase at pH 7

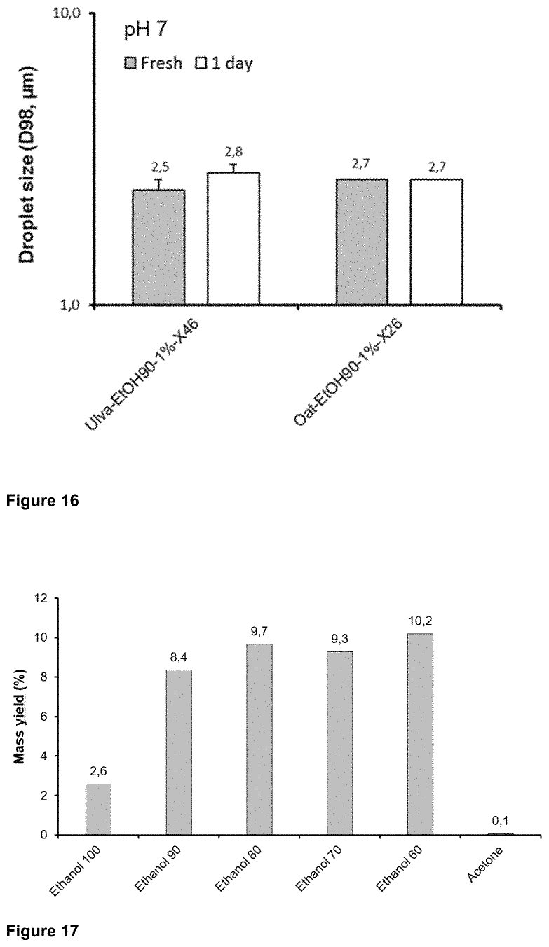

One percent of an Ulva spp. (sea lettuce) crude extract was used to stabilize oil-in-water emulsions at pH 7 made with medium chain triglycerides as the oily phase. The emulsifying activity of this extract was estimated by the droplet size (Dᵥ98) of a fresh emulsion and its variation over a day of storage (FIG. 16). The droplet size values for the Ulva spp. extract are expressed as the average of two independent replicates ±the standard deviation. The lower the droplet size, the higher the emulsifying activity. Moreover, the higher the increase of the droplet size with time, the lower the stability.

The emulsifying activity of the extract of the invention (Ulva spp.) and the stability of the emulsion they formed were further compared with those of a reference extract obtained from dehulled oat kernels, a known source of emulsifiers. The extract of the invention and the reference extract were obtained using the same solvent (ethanol:water, 90:10) and conditions of extraction and their emulsifying activities were measured with the exact same protocol. The Ulva spp. extract exhibited a significant ability to form emulsions with smaller droplet sizes (Dᵥ98 fresh=2.5 μm) than the ones obtained for the reference oat extract (2.7 μm) (FIG. 16), even though the stabilities of the emulsion after one day of storage were equivalent between the extract of the invention and the oat reference (2.8 vs. 2.7 μm, respectively).

### Example 18. Production of Crude Extracts from Dried Agarophyton chilensis by S/L Extraction

Two hundred grams of dried Agarophyton chilensis seaweed were mixed with 2 L of solvent (ethanol; ethanol:water, 90:10; ethanol:water, 80:20; ethanol:water, 70:30; ethanol:water, 60:40; and acetone) for two hours at reflux and mechanically stirred (175 rpm). The solid/liquid weight ratio was thus 1/10. The extraction medium was then filtered through an AF06 filter plate (retention rate: 15-35 μm) on a Buchner apparatus with a slight suction to remove the seaweed residues. When the residue becomes dry, the solution was let to cool down for one hour approximately. When room temperature was reached, a second filtration was done with an AF31H filter paper (retention rate: 5-12 μm) on the same system to ensure the resulting extract was devoid of any potential solid particles coming from the biological material. A rotary evaporator was then used to remove the solvent from the extract by evaporation at reduced pressure. The solid extract was freeze-dried with a dry matter typically reaching >90%. For dry matter determination, the extract was placed in an oven at 125° C. We repeated the freeze-drying step whenever the dry matter was found <90% until it overpassed this threshold.

FIG. 17 shows the mass yield obtained for the different tested solvents with maximal values for ethanol:water, 60:40 (10.2%), ethanol:water, 80:20 (9.7%), and ethanol:water, 70:30 (9.3%). The minimal yield were obtained with acetone (0.1%).

### Example 19. Polar Lipid Characterization of Crude Extracts of Agarophyton chilensis

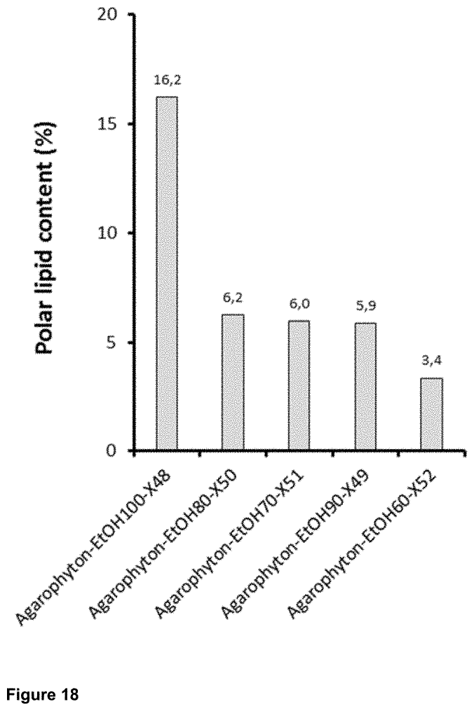

Quantification of the polar lipid was performed by reverse phase HPLC as described above in section 9 (HPLC-ELSD method to measure the polar lipid content). Results show that the highest polar lipid content (>50%) was reached for crude Agarophyton chilensis extracts obtained using ethanol as extraction solvent (FIG. 18). By contrast, crude extract obtained using ethanol:water mixtures with at least 10% of water resulted in low amounts of polar lipids (<10%).

### Example 20. The Use of 5% of an Ethanolic:Water (80:20) Crude Extract of Agarophyton chilensis as Emulsifiers in a 10% Oil-In-Water Emulsion with a Continuous Phase at pH 7

Five percents of a crude extract of Agarophyton chilensis obtained in Example 18 using an ethanol:water (80:20) mixture as extraction solvent were used to stabilize an oil-in-water emulsion at pH 7 made with medium chain triglycerides as the oily phase. The droplet size (Dᵥ98) of this emulsion was 3.4, 3.6 and 3.6 μm, just after emulsification and after one and seven days of storage, respectively. The corresponding emulsifying score (ES)—which was calculated as described above in section 12 of the material and methods—was of 5.4, which demonstrates a very good emulsifying activity because extracts with an ES equal to, or lower than, 20 are considered as emulsifying agents.

### Example 21. Production of an Ethanol:Water (80:20) Crude Extracts from Dried Alfalfa (Medicago sativa) by S/L Extraction and Use of 5% Thereof as Emulsifiers in 10% Oil-In-Water Emulsions at pH 3.5 and 7

Dried alfalfa was coarsely ground using a food machine. 200 or 150 g of each dried plant were weighed and extracted with 1200-1800 ml of solvent in a beaker. A mixture of ethanol and water (80:20 vol:vol) was used as extraction solvent. The extraction was done for 3 h at 60° C. in a glass beaker equipped with a stirrer running at 500 rpm. After filtration (Spectum EBEP-25-3-UK), the extract was filtered again using Whatman™ filter paper (CAT No 1003-110), and it was concentrated by rotary evaporator. Finally, the extracts were stored at 4° C. in a refrigerator until used. For each measurement as well as for emulsification, the extracts were adjusted to 20% solid content. A mass yield of 11.8% was obtained.

Table 5 shows the values of droplet size in alfalfa-stabilized emulsion for the ethanolic:water (80:20) alfalfa crude extract. Emulsions stable at pH 3.5 and 7 for at least 7 days could be obtained. The polar lipid content in this extract was 18%. The saponin content in this extract was of 23.6%.

TABLE 5
Droplet size obtained in emulsions prepared at pH 3.5 (emulsion
type 3) and 7 (emulsion type 4) using an ethanolic:water (80:20)
alfalfa extract at 5% in the emulsion adjusted at 20% of solid,
in fresh samples and after 7 days
Droplet size-Dv98[μm]

| | |
|---|---|
| Fresh | 7 days |

| pH 3.5 | pH 7 | pH 3.5 | pH 7 |
|---|---|---|---|
| 5.11 ± 0.11 | 5.24 ± 0.08 | 5.68 ± 0.68 | 5.65 ± 0.62 |

### Example 22. Production of an Ethanolic:Water (90:10) Crude Extract from Dried Alfalfa (Medicago sativa) by S/L Extraction and Use of 5% Thereof as Emulsifiers in a 10% Oil-In-Water Emulsion with a Continuous Phase at pH 7

Two hundred grams of dried alfalfa were mixed with 2 L of an ethanol:water (90:10) mixture for two hours at reflux and mechanically stirred (175 rpm). The extraction was performed as described above in section 3 of the material and methods. One percent of the resulting extract (C04) was then used to stabilize an oil-in-water emulsion at pH 7 made with medium chain triglycerides as the oily phase. The Dᵥ98 of this emulsion measured in duplicate was 2.3 and 2.2 just after emulsification, and 2.1 and 2.2 μm (resp.) after one day of storage. These results indicate that the crude extract had the ability to form a stable emulsion.

### Example 23. Production of Crude Extracts from Dried Microalgae Biomass by S/L Extraction

Two hundred grams of dried microalgae biomass (Isochrysis, Nannochloropsis, Tetraselmis, Chlorella sorokiniana, Chlorella vulgaris, and Chlorella zofingensis) were mixed with 2 L of solvent (ethanol; ethanol:water, 90:10; ethanol:water, 80:20; acetone; ethyl acetate, isopropanol) for two hours at reflux and mechanically stirred (175 rpm). The solid/liquid weight ratio was thus 1/10. The extraction medium was then filtered on a Buchner apparatus with a slight suction to remove the plant residues. In some specific cases, we observed a precipitate in the filtrate when the extract was let to cool down. In these cases, a second filtration was performed. For all the extracts, a rotary evaporator was used to remove the solvent from the extract by evaporation at reduced pressure. The solid extract was then freeze-dried with a dry matter typically reaching >90%. For dry matter determination, the extract was placed in an oven at 125 20° C. We repeated the freeze-drying step whenever the dry matter was found <90% until it overpassed this threshold.

### Example 24. The Use of 0.1% of Crude Extracts from Microalgae as Emulsifiers in a 10% Oil-In-Water Emulsion with a Continuous Phase at pH 7

0.1 percent of the different crude extracts from microalgae obtained in Example 23 was used to stabilize oil-in-water emulsions at pH 7 made with medium chain triglycerides as the oily phase. The emulsifying activity of these extracts was estimated by the droplet size (Dᵥ98) of a fresh emulsion and its variation after one and seven days of storage (Table 6). For most extracts, the droplet size values are expressed as the average of two independent replicates. The lower the droplet size, the higher the emulsifying activity. Moreover, the higher the increase of the droplet size with time, the lower the stability. Table 6 also shows the emulsifying scores (ES) calculated as described above in section 12 of the material and methods. Crude extracts with an ES equal to, or lower than, 20 are considered as emulsifying agents and are shown below in Table 6. The solvent of extraction is mentioned in Table 6 for all extracts. Apart from extracts obtained from Chlorella vulgaris (Z54, Z55, Z56 and Z57) and Chlorella sorokiniana (Z25 and Z26), all crude extracts have been obtained from microalgae grown photo-autotrophically.

TABLE 6
Emulsifying activity of the microalgae crude extracts (0.1%) in oil-in-water emulsion
at pH 7 as measured by the droplet size (D_{V}98) and ES. Data are sorted in decreasing
order of ES. In some cases, the polar lipid content (which have been determined as
described in section 9 of material and methods) is given in % of dry matter.

| | Droplet size (μm) | | | | |
|---|---|---|---|---|---|
| Microalgae material | Day 0 | Day 1 | Day 7 | ES | Lipid level (%) |
| Chlorella vulgaris-HPD-Filt. AF31H-EtOH90-Z57 | 9.7 | 9.8 | 36.4 | 18.9 | — |
| Chlorella vulgaris-H-Filt. AF31H-NPD-EtOH90-Z56 | 17.7 | 14.7 | 28.3 | 17.8 | — |
| Nannochloropsis-EtOH90-Z09 | 7.4 | 7.2 | 41.1 | 17.7 | — |
| Tetraselmis-EtOH90-Z39 | 6.9 | 7.6 | 24.7 | 17.4 | — |
| Chlorella vulgaris-MNPD-EtOH90-Z54 | 15.2 | 12.1 | 21.3 | 17.1 | — |
| Chlorella vulgaris-MPD-EtOH90-Z55 | 16.0 | 13.2 | 16.3 | 12.0 | 52.7 |
| Chlorella sorokiniana-M-EtOH90-Z25 | 7.5 | 7.6 | 5.5 | 8.8 | — |
| Chlorella sorokiniana-MBC-EtOH90-Z26 | 6.1 | 5.4 | 5.0 | 7.8 | 100 |
| Nannochloropsis-EtOH90-Z12 | 4.1 | 4.3 | 5.0 | 6.2 | — |

### Example 25. The Use of 1% of Crude Extracts from Microalgae as Emulsifiers in a 10% Oil-In-Water Emulsion with a Continuous Phase at pH 3.5

One percent of the different crude extracts from microalgae obtained in Example 23 was used to stabilize oil-in-water emulsions at pH 3.5 made with medium chain triglycerides as the oily phase. The emulsifying activity of these extracts was estimated by the droplet size (Dᵥ98) of the fresh emulsions and its variation over a day of storage (Table 7). The variation over a longer storage period (7 days) was also measured. Most droplet size values are expressed as the average of two independent replicates. The lower the droplet size, the higher the emulsifying activity. The higher the increase of the droplet size with time, the lower the stability. Table 7 also shows the emulsifying scores (ES) calculated as described above in section 12 of the material and methods. Crude extracts with an ES equal to, or lower than, 20 are considered as emulsifying agents and are shown below in Table 7. The solvent of extraction is mentioned in Table 7 for all extracts. Apart from extracts obtained from Chlorella vulgaris (Z54, Z55, Z56 and Z57), Chlorella sorokiniana (Z24, Z25 and Z26) and Chlorella zofingensis (Z27), all crude extracts have been obtained from microalgae grown photo-autotrophically.

TABLE 7
Emulsifying activity of the microalgae crude extracts (1%) in oil-in-water emulsion
at pH 3.5 as measured by the droplet size (D_{V}98) and ES. Data are sorted in decreasing
order of ES. In some cases, the polar lipid content (which have been determined as
described in section 9 of material and methods) is given in % of dry matter.

| | Droplet size (μm) | | | | |
|---|---|---|---|---|---|
| Microalgae material | Day 0 | Day 1 | Day 7 | Score | Polar lipids (%) |
| Isochrysis-Ethyl Acetate-Z04 | 3.7 | 32 | 184.8 | 20 | — |
| Nannochloropsis-Ethyl Acetate-Z06 | 3.1 | 15.7 | 96.4 | 19.3 | — |
| Chlorella sorokiniana-H-EtOH90-Z24 | 5.1 | 8.7 | 12.8 | 19 | — |
| Nannochloropsis-EtOH80-Z13 | 4.4 | 7.2 | — | 18.4 | — |
| Nannochloropsis-EtOH80-Z14 | 2.4 | 665.3 | 3.3 | 18.1 | — |
| Nannochloropsis-EtOH80-Z33 | 4 | 6.4 | 892.4 | 18 | — |
| Chlorella vulgaris-M-EtOH90-Z54 | 2.9 | 5.2 | 52.6 | 16.6 | — |
| Nannochloropsis-EtOH90-Z32 | 4.6 | 4.3 | 28.5 | 15.7 | 55.1 |
| Chlorella vulgaris-EtOH80-Z21 | 4.6 | 4.8 | 89.7 | 15.7 | — |
| Nannochloropsis-EtOH80-Z34 | 3.9 | 4.1 | 15 | 15 | — |
| Nannochloropsis-Acetone-Z16 | 3.6 | 3 | 593.1 | 14.6 | — |
| Chlorella sorokiniana-M-EtOH90-Z25 | 3.3 | 3.3 | 694.5 | 14.2 | — |
| Isochrysis-Acetone-Y29 | 3.2 | 3.1 | 79.5 | 14.1 | — |
| Isochrysis-iPrOH-Z02 | 2.7 | 3.2 | 26.3 | 13.3 | — |
| Nannochloropsis-EtOH90-Z09 | 4.1 | 5.7 | 4.9 | 11.5 | 73.7 |
| Tetraselmis-Ethyl Acetate-Z05 | 2.3 | 2.5 | 3.8 | 9 | — |
| Nannochloropsis-EtOH80-Y27 | 6.2 | 5.7 | 4.1 | 7.9 | — |
| Isochrysis-EtOH90-Z37 | 5.1 | 5.2 | 5 | 7 | 53.4 |
| Isochrysis-EtOH90-Y23 | 4.6 | 5.5 | 5.8 | 6.6 | 29.3 |
| Nannochloropsis-EtOH80-Z17 | 4.2 | 4.8 | 5.9 | 6.2 | — |
| Chlorella sorokiniana-MBC-EtOH90-Z26 | 4.2 | 4.6 | 4.5 | 6.2 | 100 |
| Chlorella vulgaris-H-Filt. AF31H-EtOH90-Z56 | 4.1 | 3.4 | 3.4 | 6.2 | — |
| Isochrysis-EtOH100-Y20 | 4 | 4.5 | 5.3 | 6.1 | — |
| Nannochloropsis-EtOH90-Z12 | 3.8 | 4.3 | 4.4 | 5.7 | — |
| Chlorella vulgaris-EtOH100-Z19 | 3.6 | 3 | 3.6 | 5.6 | — |
| Chlorella zofingensis-M-EtOH90-Z27 | 3.4 | 3.6 | 3.3 | 5.3 | 54.8 |
| Tetraselmis-Acetone-Y31 | 3.3 | 3.4 | 4 | 5.1 | — |
| Nannochloropsis-iPrOH-Z01 | 3.2 | 3.4 | 3.4 | 5.1 | — |
| Chlorella vulgaris-HPD-Filt. AF31H-EtOH90-Z57 | 2.8 | 2.8 | 2.8 | 4.5 | — |
| Nannochloropsis-EtOH90-Y24 | 2.8 | 3 | 3.1 | 4.4 | — |
| Nannochloropsis-iPrOH-Z29 | 2.8 | 2.7 | 2.9 | 4.4 | — |
| Nannochloropsis-EtOH100-Z08 | 2.6 | 2.7 | 3.2 | 4.2 | — |
| Nannochloropsis-iPrOH-Z30 | 2.6 | 2.8 | 3.1 | 4.1 | — |
| Chlorella vulgaris-MPD-EtOH90-Z55 | 2.6 | 2.5 | 2.6 | 4.1 | 52.7 |
| Nannochloropsis-EtOH100-Z07 | 2.3 | 2.9 | 2.4 | 3.6 | — |
| Tetraselmis-EtOH80-Y28 | 2.2 | 2.2 | 2.1 | 3.5 | — |
| Tetraselmis-EtOH90-Z39 | 2.2 | 2 | 2.4 | 3.4 | — |
| Tetraselmis-EtOH100-Y22 | 1.9 | 1.9 | 1.8 | 2.8 | — |
| Tetraselmis-EtOH90-Y25 | 1.5 | 1.6 | 1.6 | 1.9 | 69.4 |
| Nannochloropsis-EtOH100-Y21 | 1.3 | 1.4 | 1 | 1.2 | |
| Tetraselmis-iPrOH-Z03 | 1.2 | 1.2 | 1.2 | 0.8 | 98.4 |

### Example 26. The Use of 1% of Crude Extracts from Microalgae as Emulsifiers in a 10% Oil-In-Water Emulsion with a Continuous Phase at pH 7

One percent of the different crude extracts from microalgae obtained in Example 23 was used to stabilize oil-in-water emulsions at pH 7 made with medium chain triglycerides as the oily phase. The emulsifying activity of these extracts was estimated by the droplet size (Dᵥ98) of the fresh emulsions and its variation over a day of storage (Table 8). The variation over a longer storage period (7 days) was also measured for selected extracts. Most droplet size values are expressed as the average of two independent replicates. The lower the droplet size, the higher the emulsifying activity. The higher the increase of the droplet size with time, the lower the stability. Table 8 also shows the emulsifying scores (ES) calculated as described above in section 12 of the material and methods. Crude extracts with an ES equal to, or lower than, 20 are considered as emulsifying agents and are shown below in Table 8. The solvent of extraction is mentioned in Table 8 for all extracts. Apart from extracts obtained from Chlorella vulgaris (Z19, Z21, Z55, Z56 and Z57), Chlorella sorokiniana (Z25 and Z26) and Chlorella zofingensis (Z27), all crude extracts have been obtained from microalgae grown photo-autotrophically.

TABLE 8
Emulsifying activity of the microalgae crude extracts (1%) in oil-in-water emulsion
at pH 7 as measured by the droplet size (D_{V}98) and ES. Data are sorted in decreasing
order of ES. In some cases, the polar lipid content (which have been determined as
described in section 9 of material and methods) is given in % of dry matter.

| | Droplet size (μm) | | | | |
|---|---|---|---|---|---|
| Microalgae material | Day 0 | Day 1 | Day 7 | Score | Polar lipids (%) |
| Chlorella sorokiniana-M-EtOH90-Z25 | 3.4 | 752.2 | — | 19.6 | — |
| Nannochloropsis-Acetone-Y30 | 3.3 | 13.5 | — | 19.5 | — |
| Nannochloropsis-EtOH80-Z14 | 3.1 | 923.8 | — | 19.2 | — |
| Nannochloropsis-Ethyl Acetate-Z40 | 3.0 | 327.3 | 28.0 | 19.1 | — |
| Chlorella vulgaris-HPD-Filt. AF31H-EtOH90-Z57 | 52.9 | 3.6 | 3.7 | 17.2 | — |
| Nannochloropsis-EtOH80-Z13 | 5.6 | 6.6 | 15.2 | 16.5 | — |
| Chlorella sorokiniana-MBC-EtOH90-Z26 | 4.0 | 4.0 | 686.2 | 15.0 | 100 |
| Chlorella vulgaris-EtOH80-Z21 | 3.8 | 3.8 | 428.0 | 14.8 | — |
| Isochrysis-Ethyl Acetate-Z04 | 3.4 | 4.2 | 58.0 | 14.4 | — |
| Nannochloropsis-Acetone-Z15 | 2.9 | 2.6 | 24.7 | 13.7 | — |
| Isochrysis-Acetone-Y29 | 2.9 | 2.8 | 106.4 | 13.6 | — |
| Tetraselmis-Acetone-Y31 | 2.9 | 3.0 | 354.3 | 13.6 | — |
| Isochrysis-iPrOH-Z02 | 2.7 | 2.6 | 10.7 | 13.4 | — |
| Nannochloropsis-EtOH90-Z32 | 4.7 | 4.4 | 6.5 | 12.0 | 55.1 |
| Nannochloropsis-EtOH100-Z07 | 1.8 | 1.7 | 705.0 | 11.5 | — |
| Isochrysis-EtOH90-Z37 | 6.0 | 5.9 | 6.6 | 7.8 | 53.4 |
| Isochrysis-EtOH90-Y23 | 5.9 | 7.0 | 7.2 | 7.7 | 29.3 |
| Nannochloropsis-EtOH90-Z31 | 4.1 | 4.4 | 4.5 | 6.1 | — |
| Isochrysis-EtOH100-Y20 | 4.0 | 4.3 | 4.6 | 6.0 | — |
| Nannochloropsis-EtOH90-Z09 | 3.7 | 4.2 | 4.1 | 5.7 | 73.7 |
| Chlorella vulgaris-EtOH100-Z19 | 3.7 | 3.0 | 3.7 | 5.6 | — |
| Nannochloropsis-EtOH80-Z17 | 3.6 | 3.7 | 4.0 | 5.6 | — |
| Tetraselmis-Ethyl Acetate-Z05 | 3.5 | 4.2 | 4.1 | 5.4 | — |
| Chlorella vulgaris-H-filtered AF31H-EtOH90-Z56 | 3.5 | 3.5 | 3.4 | 5.4 | — |
| Nannochloropsis-EtOH100-Y21 | 3.4 | 1.0 | 0.9 | 5.3 | — |
| Chlorella zofingensis-M-EtOH90-Z27 | 3.4 | 3.4 | 3.3 | 5.3 | 54.8 |
| Nannochloropsis-EtOH80-Z34 | 3.4 | 3.4 | 3.6 | 5.3 | — |
| Nannochloropsis-EtOH80-Z33 | 3.3 | 4.2 | 4.7 | 5.2 | — |
| Nannochloropsis-EtOH80-Z36 | 2.7 | 2.7 | 2.8 | 4.4 | — |
| Nannochloropsis-EtOH90-Z12 | 2.6 | 2.8 | 2.9 | 4.2 | — |
| Chlorella vulgaris-MPD-EtOH90-Z55 | 2.4 | 2.5 | 2.5 | 3.8 | 52.7 |
| Tetraselmis-EtOH90-Z39 | 2.1 | 2.1 | 2.1 | 3.3 | — |
| Nannochloropsis-iPrOH-Z01 | 2.1 | 2.4 | 2.3 | 3.3 | — |
| Nannochloropsis-iPrOH-Z30 | 1.8 | 1.8 | 1.8 | 2.6 | — |
| Nannochloropsis-EtOH100-Z08 | 1.8 | 1.9 | 1.9 | 2.6 | — |
| Tetraselmis-EtOH90-Y25 | 1.4 | 1.5 | 1.5 | 1.4 | 69.4 |
| Tetraselmis-iPrOH-Z03 | 1.0 | 1.0 | 1.0 | 0.2 | 98.4 |
| Tetraselmis-EtOH100-Y22 | 0.9 | 1.0 | 1.0 | −0.3 | — |

### Example 27. The Use of 5% of Crude Extracts from Microalgae as Emulsifiers in a 10% Oil-In-Water Emulsion with a Continuous Phase at pH 3.5

Five percents of the different crude extracts from microalgae obtained in Example 23 were used to stabilize oil-in-water emulsions at pH 3.5 made with medium chain triglycerides as the oily phase. The emulsifying activity of these extracts was estimated by the droplet size (Dᵥ98) of the fresh emulsions and its variation over a day of storage (Table 9). The variation over a longer storage period (7 days) was also measured. Most droplet size values are expressed as the average of two independent replicates. The lower the droplet size, the higher the emulsifying activity. The higher the increase of the droplet size with time, the lower the stability. Table 9 also shows the emulsifying scores (ES) calculated as described above in section 12 of the material and methods. Crude extracts with an ES equal to, or lower than, 20 are considered as emulsifying agents and are shown below in Table 9. The solvent of extraction is mentioned in Table 9 for all extracts. Apart from extracts obtained from Chlorella vulgaris (Z55, Z56 and Z57) and Chlorella sorokiniana (Z24 and Z25), all crude extracts have been obtained from microalgae grown photo-autotrophically.

TABLE 9
Emulsifying activity of the microalgae crude extracts (5%) in oil-in-water emulsion
at pH 3.5 as measured by the droplet size (D_{V}98) and ES. Data are sorted in decreasing
order of ES. In some cases, the polar lipid content (which have been determined as
described in section 9 of material and methods) is given in % of dry matter.

| | Droplet size (μm) | | | | |
|---|---|---|---|---|---|
| Microalgae material | Day 0 | Day 1 | Day 7 | Score | Lipid content (%) |
| Nannochloropsis-Ethyl Acetate-Z06 | 3.62 | 11.77 | 27.78 | 19.9 | — |
| Nannochloropsis-Acetone-Z18 | 3.11 | 144.87 | — | 19.2 | — |
| Tetraselmis-Ethyl Acetate-Z05 | 2.57 | 53.12 | 1.04 | 18.4 | — |
| Nannochloropsis-Acetone-Y30 | 3.39 | 6.65 | 1.44 | 17.2 | — |
| Nannochloropsis-Acetone-Z15 | 2.67 | 2.22 | 24.02 | 13.3 | — |
| Isochrysis-Ethyl Acetate-Z04 | 1.87 | 2.08 | 16.04 | 11.7 | — |
| Chlorella sorokiniana-M-EtOH90-Z25 | 4.37 | 4.9 | 5.78 | 11.7 | — |
| Nannochloropsis-EtOH100-Y21 | 11 | 13.77 | 0.53 | 10.4 | — |
| Nannochloropsis-iPrOH-Z29 | 1.24 | 1.26 | 254.71 | 10.0 | — |
| Nannochloropsis-EtOH100-Z08 | 2.89 | 2.85 | 4.8 | 9.9 | — |
| Chlorella vulgaris-MPD-EtOH90-Z55 | 8.79 | 10.18 | 5.34 | 9.4 | 52.7 |
| Chlorella vulgaris-HPD-Filt. AF31H-EtOH90- | 8.46 | 8.36 | 8 | 9.3 | — |
| Tetraselmis-EtOH100-Y22 | 6.76 | 6.7 | 5.02 | 8.3 | — |
| Chlorella sorokiniana-H-EtOH90-Z24 | 6.29 | 6.15 | 1.86 | 8.0 | — |
| Isochrysis-EtOH80-Y26 | 5.86 | 5.82 | 5.36 | 7.7 | — |
| Chlorella vulgaris-H-Filt. AF31H-EtOH90-Z56 | 4.91 | 5.72 | 5.93 | 6.9 | — |
| Isochrysis-iPrOH-Z02 | 1.34 | 1.84 | 2.03 | 6.6 | — |
| Isochrysis-EtOH100-Y20 | 1.1 | 1.28 | 2.01 | 5.7 | — |
| Nannochloropsis-EtOH100-Z07 | 3.4 | 3.52 | 3.32 | 5.3 | — |
| Chlorella vulgaris-EtOH100-Z19 | 2.98 | 2.71 | 3.34 | 4.7 | — |
| Isochrysis-EtOH90-Y23 | 2.26 | 2.35 | 2.49 | 3.5 | 29.3 |
| Isochrysis-EtOH80-Z38 | 1.83 | 1.95 | 1.95 | 2.6 | — |
| Nannochloropsis-iPrOH-Z01 | 1.69 | 1.77 | 1.01 | 2.3 | — |
| Tetraselmis-EtOH80-Y28 | 1.64 | 1.69 | 1.73 | 2.1 | — |
| Isochrysis-EtOH90-Z37 | 1.59 | 1.77 | 1.84 | 2.0 | 53.4 |
| Isochrysis-Acetone-Y29 | 1.42 | 1.41 | 1.54 | 1.5 | — |
| Tetraselmis-EtOH90-Y25 | 1.12 | 0.96 | 0.96 | 0.5 | 69.4 |
| Tetraselmis-EtOH90-Z39 | 1.07 | 1.12 | 1.29 | 0.3 | — |
| Nannochloropsis-iPrOH-Z30 | 0.88 | 0.89 | 0.86 | −0.6 | — |
| Tetraselmis-iPrOH-Z03 | 0.81 | 0.73 | 0.71 | −0.9 | 98.4 |

### Example 28. The Use of 5% of Crude Extracts from Microalgae as Emulsifiers in a 10% Oil-In-Water Emulsion with a Continuous Phase at pH 7

Five percents of the different crude extracts from microalgae obtained in Example 23 were used to stabilize oil-in-water emulsions at pH 7 made with medium chain triglycerides as the oily phase. The emulsifying activity of these extracts was estimated by the droplet size (Dᵥ98) of the fresh emulsions and its variation over a day of storage (Table 10). The variation over a longer storage period (7 days) was also measured. Most droplet size values are expressed as the average of two independent replicates. The lower the droplet size, the higher the emulsifying activity. The higher the increase of the droplet size with time, the lower the stability. Table 10 also shows the emulsifying scores (ES) calculated as described above in section 12 of the material and methods. Crude extracts with an ES equal to, or lower than, 20 are considered as emulsifying agents and are shown below in Table 10. The solvent of extraction is mentioned in Table 10 for all extracts. Apart from extracts obtained from Chlorella vulgaris (Z54, Z55, Z56 and Z57) and Chlorella sorokiniana (Z24 and Z25), all crude extracts have been obtained from microalgae grown photo-autotrophically.

TABLE 10
Emulsifying activity of the microalgae crude extracts (5%) in oil-in-water emulsion
at pH 7 as measured by the droplet size (D_{V}98) and ES. Data are sorted in decreasing
order of ES. In some cases, the polar lipid content (which have been determined as
described in section 9 of material and methods) is given in % of dry matter.

| | Droplet size | | | | |
|---|---|---|---|---|---|
| Microalgae material | Day 0 | Day 1 | Day 7 | ES | Lipid content (%) |
| Chlorella sorokiniana-M-EtOH90-Z25 | 6.33 | 8.53 | 15.42 | 20.0 | — |
| Chlorella vulgaris-M-EtOH90-Z54 | 3.25 | 31.56 | 49.47 | 19.4 | — |
| Nannochloropsis-Ethyl Acetate-Z06 | 2.85 | 17.6 | — | 18.9 | — |
| Chlorella vulgaris-EtOH90-Z20 | 7.14 | 8.45 | 100.23 | 17.6 | — |
| Chlorella vulgaris-HPD-Filt. AF31H-EtOH90-Z57 | 13.96 | 14.19 | 18.89 | 16.7 | — |
| Tetraselmis-Acetone-Y31 | 40.16 | 4.9 | 0.88 | 16.0 | — |
| Isochrysis-Acetone-Y29 | 1.43 | 2.32 | 3.44 | 13.5 | — |
| Tetraselmis-Ethyl Acetate-Z05 | 2.37 | 2.32 | 23.7 | 12.8 | — |
| Isochrysis-iPrOH-Z02 | 1.83 | 2.14 | 4.05 | 11.7 | — |
| Nannochloropsis-Acetone-Z15 | 1.83 | 1.81 | 4.91 | 11.7 | — |
| Chlorella vulgaris-EtOH100-Z19 | 3.9 | 3.86 | 5.68 | 11.2 | — |
| Chlorella vulgaris-MPD-EtOH90-Z55 | 11.86 | 14.67 | 10.32 | 10.7 | 52.7 |
| Nannochloropsis-Acetone-Y30 | 2.15 | 2.38 | 3.76 | 8.6 | — |
| Chlorella sorokiniana-H-EtOH90-Z24 | 6.59 | 6.43 | 2.77 | 8.2 | — |
| Chlorella vulgaris-H-Filt, AF31H-EtOH90-Z56 | 5.16 | 4.51 | 5.71 | 7.1 | — |
| Isochrysis-EtOH100-Y20 | 1.46 | 1.67 | 2.16 | 6.9 | — |
| Isochrysis-EtOH80-Y26 | 4.63 | 5.06 | 4.58 | 6.7 | — |
| Nannochloropsis-EtOH100-Z07 | 3.81 | 4.44 | 4.09 | 5.8 | — |
| Nannochloropsis-EtOH100-Z08 | 3.14 | 3.29 | 3 | 5.0 | — |
| Isochrysis-EtOH90-Y23 | 1.79 | 1.95 | 2.08 | 2.5 | 29.3 |
| Isochrysis-EtOH90-Z37 | 1.75 | 1.9 | 1.94 | 2.4 | 53.4 |
| Tetraselmis-EtOH80-Y28 | 1.71 | 1.75 | 1.84 | 2.3 | — |
| Isochrysis-EtOH80-Z38 | 1.63 | 1.68 | 1.74 | 2.1 | — |
| Tetraselmis-EtOH100-Y22 | 1.42 | 1.37 | 1.18 | 1.5 | — |
| Nannochloropsis-iPrOH-Z01 | 1.28 | 1.32 | 1.15 | 1.1 | — |
| Tetraselmis-EtOH90-Y25 | 1.04 | 0.96 | 0.95 | 0.2 | 69.4 |
| Tetraselmis-EtOH90-Z39 | 1.04 | 1.11 | 1.28 | 0.2 | — |
| Nannochloropsis-iPrOH-Z29 | 0.9 | 0.89 | 0.88 | −0.5 | — |
| Tetraselmis-iPrOH-Z03 | 0.84 | 0.76 | 0.71 | −0.8 | 98.4 |
| Nannochloropsis-iPrOH-Z30 | 0.78 | 0.77 | 0.78 | −1.1 | — |

### Example 29. Production of a Crude Extract from Dried Dunaliella salina Microalgae by S/L Extraction and Use of 1 and 5% Thereof as Emulsifiers in a 10% Oil-In-Water Emulsion with a Continuous Phase at pH 7

Two hundred grams of dried Dunaliella salina were mixed with 2 L of an ethanol:water (90:10) mixture for two hours at reflux and mechanically stirred (175 rpm). The extraction was performed as described above in section 3 of the material and methods. One percent of the resulting extract (C03) were then used to stabilize an oil-in-water emulsion at pH 7 made with medium chain triglycerides as the oily phase. The Dᵥ98 of this emulsion measured in duplicate was 2.4 and 2.4 μm just after emulsification, and still 2.4 and 2.4 μm after one day of storage, indicating that the crude extract had the ability to form a stable emulsion.

### Example 30. Production of a Crude Extract from Dried Black Tea Leaves (Camellia sinensis) by S/L Extraction and Use of 1% Thereof as Emulsifiers in a 10% Oil-In-Water Emulsion with a Continuous Phase at pH 7

Two hundred grams of dried black tea leaves were mixed with 2 L of an ethanol:water (90:10) mixture for two hours at reflux and mechanically stirred (175 rpm). The extraction was performed as described above in section 3 of the material and methods. One percent of the resulting extract (Z58) was then used to stabilize an oil-in-water emulsion at pH 7 made with medium chain triglycerides as the oily phase. The Dᵥ98 of this emulsion was 11.4 μm just after emulsification, indicating that the crude extract had the ability to form an emulsion. A quantification of the polar lipid content has been achieved on the extract as described in section 9 and gave a value of 6.9%.

### Example 31. Production of a Crude Extract from Dried Rosemary Leaves (Rosmarinus officinalis) by S/L Extraction and Use of 5% Thereof as Emulsifiers in a 10% Oil-In-Water Emulsion with a Continuous Phase at pH 7

Two hundred grams of dried rosemary leaves were mixed with 2 L of an ethanol:water (90:10) mixture for two hours at reflux and mechanically stirred (175 rpm). The extraction was performed as described above in section 3 of the material and methods. Five percents of the resulting extract (C02) was then used to stabilize an oil-in-water emulsion at pH 7 made with medium chain triglycerides as the oily phase. The Dᵥ98 of this emulsion was 5.11 and 3.9 μm just after emulsification and after one day of storage, respectively, indicating that the crude extract had the ability to form a stable emulsion.

### Example 32. Production of a Crude Extract from Dried Green Pea Pods (Pisum sativum) by S/L Extraction and Use of 1% Thereof as Emulsifiers in a 10% Oil-In-Water Emulsion with a Continuous Phase at pH 7

Two hundred grams of dried green pea pods were mixed with 2 L of an ethanol:water (90:10) mixture for two hours at reflux and mechanically stirred (175 rpm). The extraction was performed as described above in section 3 of the material and methods. One percent of the resulting extract (Z67) was then used to stabilize an oil-in-water emulsion at pH 7 made with medium chain triglycerides as the oily phase. The Dᵥ98 of this emulsion was 5.7 μm just after emulsification, indicating that the crude extract had the ability to form an emulsion.

### Example 33. Production of a Crude Extract from Fresh Spring Onion (Allium fistulosum) Biomass by a Direct S/L Extraction and Use of 1% Thereof as Emulsifiers in a 10% Oil-In-Water Emulsion with a Continuous Phase at pH 3.5

One litre of ethanol was heated to 70° C. One hundred grams of fresh spring onion were weighted and properly mixed with a classic cooking blender (Moulinex, France) during a few min. The plant was then added to the hot solvent and the extraction was performed as described above in section 4 (Direct S/L extraction on fresh materials).

One percent of the resulting extract (Z42 green) was then used to stabilize an oil-in-water emulsion at pH 3.5 made with medium chain triglycerides as the oily phase. The Dᵥ98 of this emulsion was 3.5, 3.1 and 164.9 μm, just after emulsification and after one and seven days of storage, respectively. The corresponding ES—which was calculated as described above in section 12 of the material and methods— was of 14.5, which demonstrates a notable emulsifying activity, at least after one day of storage, because extracts with an ES equal to, or lower than, 20 are considered as emulsifying agents. The droplet size increase after the seventh day of storage is likely to be solvable by a simple increase of the emulsion's viscosity.

### Example 34. Production of a Crude Extract from Fresh Broccoli Rabe (Brassica ruvo) Biomass by an Indirect S/L Extraction and Use of 1% Thereof as Emulsifiers in a 10% Oil-In-Water Emulsion with a Continuous Phase at pH 3.5

One litre of ethanol was heated to 70° C. One hundred grams of fresh broccoli rabe were weighted and properly mixed with a classic cooking blender (Moulinex, France) during a few min. The plant was then added to the hot solvent and the extraction was performed as described above in section 5 (Indirect S/L extraction on fresh materials).

One percent of the resulting extract (Z49) was then used to stabilize an oil-in-water emulsion at pH 3.5 made with medium chain triglycerides as the oily phase. The Dᵥ98 of this emulsion was 3.6 μm just after emulsification, demonstrating that the extract was able to form an emulsion.

### Example 35. Production of a Crude Extract from Fresh Carrot Leaves (Daucus carota) Biomass by an Indirect S/L Extraction and Use of 1% Thereof as Emulsifiers in a 10% Oil-In-Water Emulsion with a Continuous Phase at pH 7

One litre of ethanol was heated to 70° C. One hundred grams of fresh carrot leaves were weighted and properly mixed with a classic cooking blender (Moulinex, France) during a few min. The plant was then added to the hot solvent and the extraction was performed as described above in section 5 (Indirect S/L extraction on fresh materials).

One percent of the resulting extract (Z51) was then used to stabilize an oil-in-water emulsion at pH 7 made with medium chain triglycerides as the oily phase. The Dᵥ98 of this emulsion was 5.2 μm just after emulsification, demonstrating that the extract was able to form an emulsion.

### Example 36. Production of Crude Extracts from Fresh Spinach Leaves by an Indirect S/L Extraction and Use of 1% Thereof as Emulsifiers in a 10% Oil-In-Water Emulsion with a Continuous Phase at pH 3.5

One litre of ethanol was heated to 70° C. The residue was recovered and added to the hot solvent to proceed to the extraction as described above in section 5 (Indirect S/L extraction on fresh materials). The resulting crude extract is further referred to as extract Z48.

In another extraction, 250 g of the same fresh spinach leaves were juiced using a cooking juice-extractor device at room temperature for 5 min, then the juice was clarified by filtration, and the resulting filtration cake was pooled with the recovered residue from the juice-extractor, and pressed using a hydraulic press at 15 bar to remove as much residual juice as possible. The dry cake was then extracted as described above in section 5 (Indirect S/L extraction on fresh materials). The resulting crude extract is further referred to as extract Z69.

One percent of both crude extracts was then used to stabilize an oil-in-water emulsion at pH 3.5 made with medium chain triglycerides as the oily phase.

For extract Z48, the Dᵥ98 of this emulsion was 4.6, 5.6 and 40.2 μm, just after emulsification and after one and seven days of storage, respectively. The corresponding ES—which was calculated as described above in section 12 of the material and methods— was of 14.5, which demonstrates a notable emulsifying activity, at least after one day of storage, because extracts with an ES equal to, or lower than, 20 are considered as emulsifying agents.

For extract Z69, the emulsifying performances were slightly better with a Dᵥ98 of the emulsion of 3.6, 3.4 and 21 μm, just after emulsification and after one and seven days of storage, respectively. The corresponding ES was of 14.6.

In both cases, the droplet size increase after the seventh day of storage is likely to be solvable by a simple increase of the emulsion's viscosity.

### Example 37. Production of a Crude Extract from Fresh Spring Onion (Allium fistulosum) Biomass by a Direct S/L Extraction and Use of 1% Thereof as Emulsifiers in a 10% Oil-In-Water Emulsion with a Continuous Phase at pH 7

One litre of ethanol was heated to 70° C. One hundred grams of fresh spring onion were weighted and properly mixed with a classic cooking blender (Moulinex, France) during a few min. The plant was then added to the hot solvent and the extraction was performed as described above in section 4 (Direct S/L extraction on fresh materials).

One percent of the resulting extract (Z42 green) was then used to stabilize an oil-in-water emulsion at pH 7 made with medium chain triglycerides as the oily phase. The Dᵥ98 of this emulsion was 3.3, 3.2 and 3.0 μm, just after emulsification and after one and seven days of storage, respectively. The corresponding ES—which was calculated as described above in section 12 of the material and methods— was of 5.1, which demonstrates a very good emulsifying activity, because extracts with an ES equal to, or lower than, 20 are considered as emulsifying agents.

### Example 38. Production of a Crude Extract from Fresh Celery Leaves (Apium graveolens) Biomass by a Direct S/L Extraction and Use of 1% Thereof as Emulsifiers in a 10% Oil-In-Water Emulsion with a Continuous Phase at pH 7

One litre of ethanol was heated to 70° C. One hundred grams of fresh celery leaves were weighted and properly mixed with a classic cooking blender (Moulinex, France) during a few min. The plant was then added to the hot solvent and the extraction was performed as described above in section 4 (Direct S/L extraction on fresh materials). One percent of the resulting extract (Z52) was then used to stabilize an oil-in-water emulsion at pH 7 made with medium chain triglycerides as the oily phase. The Dᵥ98 of this emulsion was 10.5 μm, just after emulsification, indicating that the extract was able to form an emulsion.

### Example 39. Production of a Crude Extract from Fresh Broccoli Rabe (Brassica ruvo) Biomass by a Direct S/L Extraction and Use of 1% Thereof as Emulsifiers in a 10% Oil-In-Water Emulsion with a Continuous Phase at pH 7

One litre of ethanol was heated to 70° C. One hundred grams of fresh broccoli rabe were weighted and properly mixed with a classic cooking blender (Moulinex, France) during a few min. The plant was then added to the hot solvent and the extraction was performed as described above in section 4 (Direct S/L extraction on fresh materials).

One percent of the resulting extract (Z44) was then used to stabilize an oil-in-water emulsion at pH 7 made with medium chain triglycerides as the oily phase. The Dᵥ98 of this emulsion was 4.5 μm, just after emulsification, indicating that the extract was able to form an emulsion.

### Example 40. Production of a Crude Extract from Fresh Radish Leaves (Raphanus sativus) Biomass by a Direct S/L Extraction and Use of 1% Thereof as Emulsifiers in a 10% Oil-In-Water Emulsion with a Continuous Phase at pH 7

One litre of ethanol was heated to 70° C. One hundred grams of fresh radish leaves were weighted and properly mixed with a classic cooking blender (Moulinex, France) during a few min. The plant was then added to the hot solvent and the extraction was performed as described above in section 4 (Direct S/L extraction on fresh materials).

One percent of the resulting extract (Z47) was then used to stabilize an oil-in-water emulsion at pH 7 made with medium chain triglycerides as the oily phase. The Dᵥ98 of this emulsion was 3.7 μm, just after emulsification, indicating that the extract was able to form an emulsion.

### Example 41. Production of Crude Extracts from Fresh Spinach Leaves by an Indirect S/L Extraction and Use of 1% Thereof as Emulsifiers in a 10% Oil-In-Water Emulsion with a Continuous Phase at pH 7

One litre of ethanol was heated to 70° C. Two hundred and fifty grams of fresh spinach leaves were juiced using a cooking juice-extractor device at room temperature for 5 min. The juice was clarified by filtration, and the resulting filtration cake was pooled with the recovered residue from the juice-extractor, and pressed using a hydraulic press at 15 bar to remove as much residual juice as possible. The dry cake was then extracted as described above in section 5 (Indirect S/L extraction on fresh materials). The resulting crude extract is further referred to as extract Z69.

One percent of this extract was then used to stabilize an oil-in-water emulsion at pH 7 made with medium chain triglycerides as the oily phase. The Dᵥ98 of this emulsion was 4.0, 4.3 and 6.1 μm, just after emulsification and after one and seven days of storage, respectively. The corresponding ES—which was calculated as described above in section 12 of the material and methods— was of 11.3, which demonstrates a notable emulsifying activity, because extracts with an ES equal to, or lower than, 20 are considered as emulsifying agents.

### Example 42. Production of Ethanolic:Water (90:10) Crude Extracts from Dried Spinach by S/L Extraction, Decolorization Thereof with Powdery Activated Carbon or Filter Plates Coated with Charcoal, and Use of 1 or 5% Thereof in a 10% Oil-In-Water Emulsion with a Continuous Phase at pH 3.5 or 7

For Y06, 200 g of dried spinach were mixed with 3 L of an ethanol:water (90:10) mixture for two hours at reflux and mechanically stirred (175 rpm). The extraction was performed as described above in section 3 of the material and methods (S/L ratio: 1:15). One and five percents of the resulting extract was then used to stabilize an oil-in-water emulsion at pH 3.5 and 7 made with medium chain triglycerides as the oily phase.

For Y13, the same process was applied except that before drying the filtrate of the first solvent pass, a second pass with a new ethanol:water (90:10) mixture was performed in the same conditions as the first pass (2 h at reflux under mechanically stirred at 175 rpm, S/L ratio: 1:15). One and five percents of the resulting extract was then used to stabilize the emulsion above-mentioned at pH 3.5 and 7.

For Y10 and Y15, the same one-pass extraction protocol as previously described for Y06, was applied, except that a decolorization step using powdery charcoal was performed at the end as described in section 7 of the material and methods.

For Y09, the same 2-pass extraction protocol as previously described for Y13 was applied, except that a decolorization step using powdery charcoal was performed at the end as described in section 7 of the material and methods.

For Y37, the same one-pass extraction protocol as previously described for Y06, was applied, except that a decolorization step using a R55S filter plate coated with charcoal was performed at the end as described in section 8 of the material and methods.

For Y41, the same one-pass extraction protocol as previously described for Y06, was applied, except that a decolorization step using a Filtrox filter plate coated with charcoal was performed at the end as described in section 8 of the material and methods. A quantification of the polar lipids (method described in section 9 of the material and methods) was also achieved on purified extract Y41 and found a content of 27.6%.

Color of 1% emulsions was measured by reflection in the CIELAB color space in a borosilicate tube cell with a spectrophotometer (Konica Minolta CM-5). The emulsions were given into the measurement cell at 10 mm height.

All spinach extracts were obtained from photosynthetically active parts of the plant (i.e. leaves) so that they are green. This color was still visible in the emulsions prepared with 1% extract. Emulsions prepared with Sweoat PL40 as reference (oat oil) showed no green color. Hence, we calculated the color difference ΔE* according to equation 1 with L₁*, a₁*, and b₁* being measured in the emulsion with oat oil and L₂*, a₂*, and b₂* being measured in the emulsion stabilized with an ethanolic:water (90:10) spinach.

ΔE*_{ab}=√(L*₂−L*₁)²+(a*₂−a*₁)²+(b*₂−b*₁)² Equation 1:

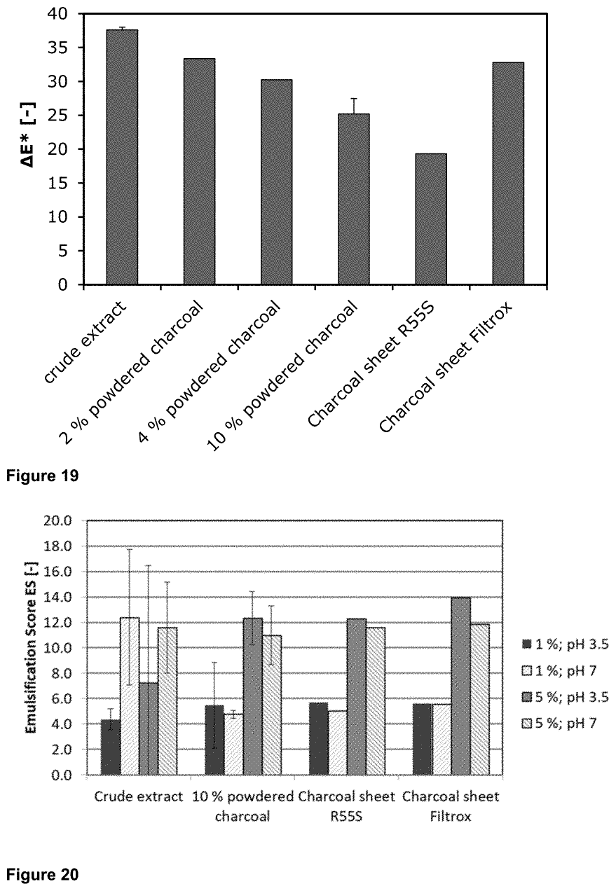

FIG. 19 shows the AE decrease as a function of the applied decolorization (purification) protocol. The fact that AE decreases with the addition of growing quantities of charcoal and with the use of the R55S filter plate means that the resulting emulsions get closer to the whitish oat oil reference. There is still a visible difference between the emulsions even after purifying them with an activated carbon filter sheet R55S but the color changes from green to yellow and the L value increases distinctly (from 67 to 87), demonstrating that the sample is more white.

In FIG. 20, it can be seen that the emulsification performance is not negatively influenced by the purification within the reproducibility of the experiments. This means that no or only little amounts of emulsifying molecules adsorbed to the activated charcoal and that filter material of the right selectivity was chosen.

### Example 43. Production of Ethanolic:Water (90:10) Crude Extracts from Dried Spirulina by S/L Extraction, Decolorization Thereof with Powdery Activated Carbon, and Use of 5% Thereof in a 10% Oil-In-Water Emulsion with a Continuous Phase at pH 3.5 or 7

For Y16, 200 g of dried spirulina were mixed with 2 L of an ethanol:water (90:10) mixture for two hours at reflux and mechanically stirred (175 rpm). The extraction was performed as described above in section 3 of the material and methods. Five percents of the resulting extract was then used to stabilize an oil-in-water emulsion at pH 3.5 and 7 made with medium chain triglycerides as the oily phase.

For Y18, the same process was applied except that before drying the filtrate of the first solvent pass, a second pass with a new ethanol:water (90:10) mixture was done in the same condition as the first pass (2 h at reflux under mechanically stirred at 175 rpm). Five percents of the resulting extract was then used to stabilize the emulsion above-mentioned at pH 3.5 and 7.

For Y17 and 19, the same protocol as previously described for Y16 and Y18, respectively, was applied, except that a decolorization step was performed as described in section 7 of the material and methods. Among these spirulina extracts, the polar lipid content of Y16 and 17 were measured as described in section 9 of the material and methods and gave values of 26.4 and 27.6%, respectively.

FIG. 21 shows that droplet size slightly increases with the purification. However, the droplet sizes achieved with the purified extract are still very small (about 1.5 μm) and the emulsions are very stable over time. Hence, the purified extracts are very good emulsifiers.

TABLE 11
Influence of the purification protocol on the colorimetric properties of spirulina extracts.
The ΔE is calculated in comparison to a reference emulsion prepared with oat oil.

| | | Purification | | | | |
|---|---|---|---|---|---|---|
| | | with 10% | | | | |
| Extract | | powdered | | | | |
| Number | Description of sample | charcoal | L* | a* | b* | ΔE |
| | Reference Sweoat PL15 | — | 94.3 | −1.0 | 5.1 | 0 |
| Y16 | Spirulina Ethanol 90%, 1 extraction pass | no | 54.6 | 0.5 | 16.0 | 41.2 |
| Y18 | Spirulina Ethanol 90%, 2 extraction passes | no | 53.4 | 0.25 | 15.9 | 42.4 |
| Y17 | Spirulina Ethanol 90%, 1 extraction pass | yes | 84.2 | −2.3 | 14.2 | 13.7 |
| Y19 | Spirulina Ethanol 90%, 2 extraction passes | yes | 81.3 | 1.9 | 18.8 | 19.1 |

Table 11 shows how L* increases and the AE-value decreases when a purification method is applied to the spirulina extract, which means the resulting emulsion gets closer to the whitish oat oil reference. There is still a visible difference between the oat oil emulsion and the emulsions after purifying the extracts with 10% charcoal but the colour perception by eye changes from green to yellow and the L value increases distinctly (from about 54 to about 83), meaning that the samples are more white.

### Example 44. Production of an Ethanolic:Water (90:10) Crude Extract from Dried Parsley Leaves (Petroselinum crispum) by S/L Extraction, Decolorization Thereof with Filter Plate Coated with Activated Charcoal, and Use of 1% Thereof in a 10% Oil-In-Water Emulsion with a Continuous Phase at pH 7

Two hundred grams of dried parsley leaves (Petroselinum crispum) were mixed with 2 L of an ethanol:water (90:10) mixture for two hours at reflux and mechanically stirred (175 rpm). The extraction and decolorization were performed as described above in section 8 of the material and methods. One percent of the resulting extract (Y45) was then used to stabilize an oil-in-water emulsion at pH 7 made with medium chain triglycerides as the oily phase. The Dᵥ98 of this emulsion was 15.8 μm just after emulsification, indicating that the crude extract had the ability to form an emulsion. A quantification of the polar lipid content has been achieved on the extract as described in section 9 and gave a value of 5.6%.

Colorimetric properties of the decolorized parsley extract was then measured before drying with a device from Spectramagic NX in transmittance mode.

Table 12 shows that the L value increases distinctly from 11 to 93, which means the sample gets less dark. The b value also changes from 19 to 53 indicating that the sample is more yellow. The constant value of a might seem surprising however the large changes of L and b can also account for a perceived decrease of green in the sample.

TABLE 12
Colorimetric properties of a crude vs.
a decolorized extract of parsley

| | L* | a* | b* |
|---|---|---|---|
| Parsley-EtOH90-Crude extract | 11.2 | −6.9 | 19.1 |
| Parsley-EtOH90-Carbon sheet-R55S | 92.6 | −6.9 | 52.9 |

### Example 45. The Use of 1% of Crude Extracts from Spinach as Emulsifiers in a 10% Water-In-Oil Emulsion with an Aqueous Phase at pH 7

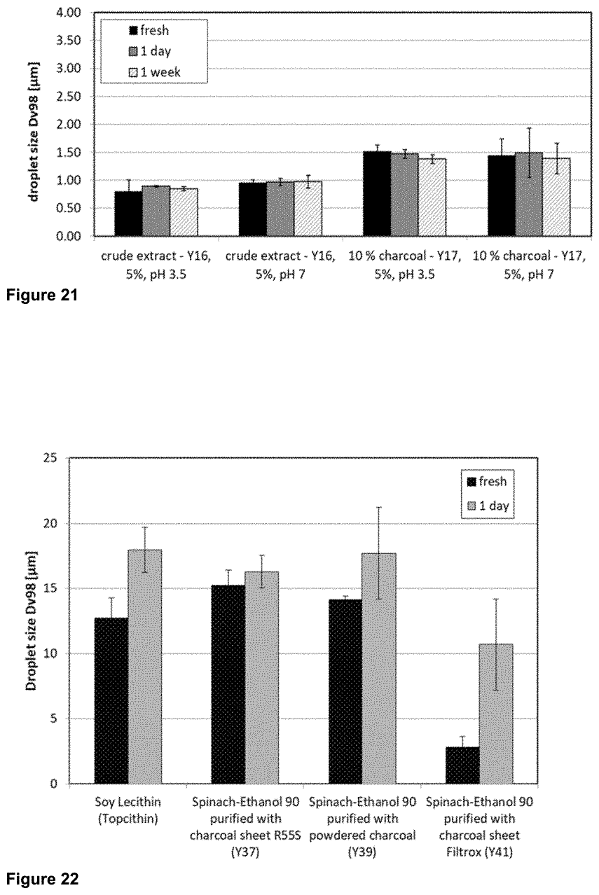

In FIG. 22 the Dᵥ98 of emulsions prepared a commercially available soy lecithin (Topcithin, Cargill) is shown in comparison with three purified plant extracts from Spinach. Surprisingly spinach extract (extracted with 90% ethanol) is not only performing well as oil-in-water emulsifier but also for water-in-oil emulsions. The initial droplet size of spinach extract purified with charcoal sheet Filtrox is distinctly smaller than for Soy Lecithin. For all purified extracts the droplet size after one day is comparable or smaller than for soy lecithin. An emulsifier so versatile to be able to stabilize water-in-oil as well as oil-in-water emulsions is highly advantageous because it can be flexible used in many different applications of different emulsion type, dispersed phase content or oils of different polarity.

### Example 46. Preparation of Coffee Creamer with 1% Purified Spinach Extract

A series of creamer emulsion according to the present invention were prepared by performing the steps of:
1. Preparing an oil phase by mixing a known amount (Table 13) of coconut oil (Kristal, AAK) and in example 37a, a known amount of Spinach extract containing polar lipids in a vessel equipped with a magnetic stirrer hotplate operating at 300 rpm and 50° C. (IKA, RET Laboratory);
2. Preparing a water phase by dissolving a known amount of pea protein (Pisane c9, Cosucra) in a known amount of water at 40° C. for 30 min while stirring with an overhead stirrer at 300 rpm.
3. Add a known amount of oat Syrup (Natu-Oat 35, Meurens) and a known amount of oat flour (Sweoat P19, Swedish Oat Fiber) to the water phase, and hydrate for another 30 min at room temperature while stirring with an overhead stirrer (Heidolph, HeiTorque) at 300 rpm
4. Combine a known amount of gellan gum (Kelcogel CG-HA, CP Kelco) with a known amount of guar gum (Keystone® 7555 Guar Gum, Main Street Ingredients) and a known amount of crystalline sugar (Magyar Cukor) and add this mix to the water phase while stirring with an overhead stirrer at 300 rpm at room temperature.
5. Heat up the water phase to 60° C., add oil phase while using Rotor-Stator mixer (Kinematica, PT-DA 3030-6060) operating at 8,000 rpm for 90 s, to obtain the pre-emulsion
6. Emulsifying the pre-emulsion by passing one time through a two-stage high-pressure homogenizer (GEA, Lab Homogenizer Panda Plus 2000), operating at a first stage valve pressure of 350 bar and a second stage valve pressure of 50 bar, in order to obtain the final emulsion.
7. Pasteurizing the final emulsion through a water bath operating at 95° C. for 12 min.

In all examples, step 5. resulted in oil-in-water emulsions.

TABLE 13
Composition of creamer emulsion

| | Ex. 37a | Ex. 37b |
|---|---|---|
| Coconut oil [wt.-%] | 7.5 | 7.5 |
| Purified Spinach extract EtOH90 [wt.-%] | 1 | 0 |
| Pea protein [wt.-%] | 0.3 | 0.3 |
| Water [wt.-%] | 60.74 | 61.74 |
| Oat syrup [wt.-%] | 9 | 9 |
| Oat flour [wt.-%] | 1.33 | 1.33 |
| Crystalline Sucrose [wt.-%] | 20 | 20 |
| Gellan Gum [wt.-%] | 0.03 | 0.03 |
| Gum Guar [wt.-%] | 0.1 | 0.1 |

TABLE 14
Droplet sizes of coffee creamers. The volume weighted mean
diameter D[4, 3] and Dv90 of the droplet size distribution
were then calculated using the software implemented in the
measurement instrument. The Dv90, is defined as the diameter
where 90% of the population (Volume) lies below this value.

| | Droplet size D[4, 3] [μm] | Droplet size d(90) [μm] |
|---|---|---|
| Example 37a, fresh | 4.62 | 7.71 |
| Example 37b, fresh | 9.76 | 18.22 |

In Table 14, it can be seen that the addition of Purified Spinach Extract led to a decrease in droplet size in the coffee creamer. This is advantageous for a longer shelf life as well as a better whitening effect in the coffee.

1. Extract rich in polar lipids obtained from microalgae, macroalgae, photosynthetic bacteria and/or photosynthetic organ(s) and/or tissue(s) of a plant and combinations thereof.

2. Extract according to claim 1, wherein the photosynthetic organ(s) and/or tissue(s) of a plant, macroalgae, microalgae and/or photosynthetic bacteria are recognized as food grade or recognized as GRAS (Generally Recognized as Safe).

3. Extract according to claim 1, wherein the photosynthetic organ(s) and/or tissue(s) is from one or more of the following plants: alfalfa, spinach, broccoli rabe, broccoli, red radish, guarana, rosemary, sage, thyme, mint, basil, Perilla frutescens, ajwain, angelica, anise, asafoetida, caraway, carrot, celery, chervil, coriander, cumin, dill, fennel, lovage, cow parsley, parsley, parsnip, sea holly, silphium oregano, lettuce, fenugreek, lentil, lupine, pea, garlic, scallion, leek, chive, and chinese onion, onions, green onions, beetroot, parsley, yerba mate, tea, endive, watercress, nettle, carrots, sweet potato, pak choi, water spinach.

4. Extract according to claim 1, wherein the microalgae is selected from one or more of Chlorella, Chysophyceae, Xantophyceae, Baccilariophyceae, Dinophyceae, Rodophyceae, Phaeophyceae, Chlorophyceae, Prasinophyceae, Cryptophyceae, Crypthecodinium, Cylindrothec, Botryococcus, Dunaliella (such as Dunaliella salina), Euglena gracilis, Isochrysis, Nannochloropsis, Neochloris, Nitzschia Scenedesmus, Chlorobotrys, Eustigmatos, Phaeodactylum, Porphyridium, Pseudostaurastrum, Schizochytrium, Tetraselmis, Vischeria, Monodopsis, Pseudocharaciopsis.

5. Extract according to claim 1, wherein the photosynthetic bacteria is selected from one or more of the following genera: Spirulina (Arthrospira, such as Arthrospira platensis, Arthrospira maxima), Limnospira (Limnospira platensis), Synechocystis, Nostoc, Cyanothece, Aphanizomenon (such as Aphanizomenon flosaquae).

6. Extract according to claim 1, wherein the macroalgae is selected from one or more of the following species: Ascophyllum nodosum, Fucus serratu, F. vesiculosus. Himanthalia elongata, Undaria pinnatifida, Laminaria digitata, L. saccharina, L. japonica, Alaria esculenta, Palmaria palmata (dulse), Porphyra umbilicalis, P. tenera, P. yezoensis, P. dioica, P. purpurea, P. laciniata, P. leucostica, Chondrus crispus, Gracilaria verrucosa, Lithothamnium calcareum, Enteromorpha spp., and Ulva spp.

7. Extract according to claim 1, wherein the extract is selected from methyltetrahydrofuran extracts or hydro methyltetrahydrofuran extracts, acetate extracts or hydro-acetate extracts, isopropanol or hydroisopropanolic extracts, acetone or hydroacetonic extracts, chloroform extracts, methanol or hydromethanolic extracts, ethanol or hydroethanolic extracts or mixtures thereof.

8. Extract according to claim 1, wherein the photosynthetic organ(s) and/or tissue(s) of a plant, macroalgae, microalgae and/or photosynthetic bacteria is a raw material and/or a spent material.

9. Extract according to claim 1 wherein the extract is selected from a Crude extract rich in polar lipids or a Purified extract rich polar lipids.

10. Crude Extract according to claim 9, wherein the extract comprises at least 5%, at least 10% of polar lipids, at least 20% of polar lipids, at least 25 wt %, at least 30%, at least 40%, at least 50%, such as at least 60% of polar lipids based on the total weight of the extract.

11. Purified extract rich in polar lipids according to claim 9, wherein the purified extract comprises at least 10% of polar lipids, at least 15 wt %, at least 20 wt %, at least 30 wt %, at least 35 wt %, at least 40 wt % at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt % or at least 99 wt % of polar lipids in based on the total weight of the purified extract.

12. Purified extract rich in polar lipids according to claim 9, wherein the purified extract was is obtained using activated carbon and/or carbon filter plate.

13. Purified extract rich in polar lipids according to claim 9 having improved organoleptic properties including such as a neutral odour, a light colour and/or absence of off-taste.

14. Purified extract rich in polar lipids according to claim 9 having a L1*, a1*, and b1* that corresponds to white or is near to white.

15. Extract according to claim 1, wherein the polar lipids phase comprises galactosyl acylglycerols, phospholipids, lysophospholipids, sulphur lipids, betain lipids and/or furan-based lipids or oxidation products thereof.

16. Extract according to claim 1, wherein the polar lipid phase comprises at least 5 wt %, at least 8 wt %, at least 10 wt %, at least 11 wt %, at least 12 wt %, at least 20%, or at least 30 wt % of galactosyl acylglycerols based on the total weight of the polar lipid phase.

17. Extract according to claim 1, wherein the polar lipid phase comprises at least 5 wt % of sulphur lipids, based on the total weight of the polar lipid fraction.

18. Purified extract rich in polar lipids according to claim 9, wherein the Purified extract contain less than 10% of sugars, proteins, peptides, chlorophylls and/or waxes.

19. (canceled)

20. (canceled)

21. Emulsion comprising at least one Extract according to claim 1 as an emulsifying agent, wherein the emulsion optionally does not comprise other emulsifying agents.

22. (canceled)

23. Emulsion according to claim 21, wherein the emulsifying agent is present in a concentration of from about 0.1 to about 10 wt %.

24. Emulsion according to claim 21, wherein the emulsion is selected from the group consisting of a water-in-oil emulsion and an oil-in-water emulsion.

25. Process for preparing an emulsion comprising:
a) mixing ingredients of an aqueous phase;
b) mixing ingredients of a lipid phase;
c) dispersing one or more Extracts according to claim 1 in one or both of the aqueous phase or the lipid phase; and
d) homogenizing the two phases to form an emulsion.

26. (canceled)

27. (canceled)

28. Emulsion according to claim 21, wherein the emulsion is free of synthetic or artificial emulsifiers and/or structuring agents.

29. Emulsion according to claim 21, wherein the emulsion has a pH from about 2 to 10, such as from about 3 to 7.

30. Emulsion according to claim 21, wherein the droplets size is comprised between 0.05 and 50 micrometres.

31. Emulsion according to claim 21, wherein the droplet size remains stable for at least one day of storage at ambient temperature (25° C.).

32. A food or beverage product for humans or animals, a nutritional supplement, a nutraceutical formulation, a fragrance or flavouring, a pharmaceutical or veterinary formulation, an oenological or cosmetic formulation comprising an emulsion according to claim 21.

33. A food or beverage product for humans or animals, a nutritional supplement, a nutraceutical formulation, a fragrance or flavouring, a pharmaceutical or veterinary formulation, an oenological or cosmetic formulation comprising at least one emulsifying extract according to claim 1.

34. Food or beverage according to claim 32 selected from sauces, mayonnaises, snacks, ice creams and desserts, dairy products, beverages, sausages and condiments, process products, meat analogues, coffee creamers, baked goods, spreads or margarines.
